SEQUENCE LISTING (110 sequences)

SEQ ID NO: 1 - protein, 15 aa, Artificial Sequence
  Synthetic
DEEEIQKAIEELLRK

SEQ ID NO: 2 - protein, 20 aa, Artificial Sequence
  Synthetic
TEDEEYEERVREALEEAKKK

SEQ ID NO: 3 - protein, 17 aa, Artificial Sequence
  Synthetic
DEEEEFKRRVQEYQEKY

SEQ ID NO: 4 - protein, 20 aa, Artificial Sequence
  Synthetic
DKEDELRELAKELVREYEKR

SEQ ID NO: 5 - protein, 20 aa, Artificial Sequence
  Synthetic
TEEERLREEVKRVAQRAEEE

SEQ ID NO: 6 - protein, 20 aa, Artificial Sequence
  Synthetic
DLEERVRKAVNEAQEEAKRN

SEQ ID NO: 7 - protein, 20 aa, Artificial Sequence
  Synthetic
DEREEEQRRRLEEVKEEAKR

SEQ ID NO: 8 - protein, 20 aa, Artificial Sequence
  Synthetic
DEEEEEKEEIRRQLDEARER

SEQ ID NO: 9 - protein, 12 aa, Artificial Sequence
  Synthetic
EEEAAIIIAQRF

SEQ ID NO: 10 - protein, 12 aa, Artificial Sequence
  Synthetic
EEEAAIIIVQRF

SEQ ID NO: 11 - protein, 12 aa, Artificial Sequence
  Synthetic
EEEAAIIIVQTF

SEQ ID NO: 12 - protein, 12 aa, Artificial Sequence
  Synthetic
EELVAQIALLIL

SEQ ID NO: 13 - protein, 11 aa, Artificial Sequence
  Synthetic
DERDAEAALRA

SEQ ID NO: 14 - protein, 12 aa, Artificial Sequence
  Synthetic
EEEIALLVALAA

SEQ ID NO: 15 - protein, 12 aa, Artificial Sequence
  Synthetic
EEEIAQILLLAL

SEQ ID NO: 16 - protein, 12 aa, Artificial Sequence
  Synthetic
ENEAALEAALRL

SEQ ID NO: 17 - protein, 12 aa, Artificial Sequence
  Synthetic
EQDLAVLYLEAV

SEQ ID NO: 18 - protein, 13 aa, Artificial Sequence
  Synthetic
DEEEAAIEVAQNF

SEQ ID NO: 19 - protein, 14 aa, Artificial Sequence
  Synthetic
ERQAKHISEYIRRY

SEQ ID NO: 20 - protein, 14 aa, Artificial Sequence
  Synthetic
ERQGKHISEYIRRY

SEQ ID NO: 21 - protein, 14 aa, Artificial Sequence
  Synthetic
EEAAKRAAQYVRER

SEQ ID NO: 22 - protein, 13 aa, Artificial Sequence
  Synthetic
EEAERIAQEIRDR

SEQ ID NO: 23 - protein, 14 aa, Artificial Sequence
  Synthetic
EEQAERAAKYIKKK

SEQ ID NO: 24 - protein, 14 aa, Artificial Sequence
  Synthetic
EEQAERVAQYIREV

SEQ ID NO: 25 - protein, 14 aa, Artificial Sequence
  Synthetic
EEQAERVAKYVHKL

SEQ ID NO: 26 - protein, 14 aa, Artificial Sequence
  Synthetic
EEEAKRVADIVKKL

SEQ ID NO: 27 - protein, 14 aa, Artificial Sequence
  Synthetic
EEEAERVAEYVRRE

SEQ ID NO: 28 - protein, 15 aa, Artificial Sequence
  Synthetic
EELRKRVWEAAQKAY

SEQ ID NO: 29 - protein, 15 aa, Artificial Sequence
  Synthetic
HELRKRVWEAAQKAY

SEQ ID NO: 30 - protein, 15 aa, Artificial Sequence
  Synthetic
DELNKRVWEAAQKAY

SEQ ID NO: 31 - protein, 15 aa, Artificial Sequence
  Synthetic
HELSTRVWEAAQKAY

SEQ ID NO: 32 - protein, 15 aa, Artificial Sequence
  Synthetic
HELSQRVWEAAQKAY

SEQ ID NO: 33 - protein, 15 aa, Artificial Sequence
  Synthetic
REDQEAAWRAAQEAM

SEQ ID NO: 34 - protein, 14 aa, Artificial Sequence
  Synthetic
EAAERVWKIAQEAL

SEQ ID NO: 35 - protein, 15 aa, Artificial Sequence
  Synthetic
REDERIAWELAQEAQ

SEQ ID NO: 36 - protein, 15 aa, Artificial Sequence
  Synthetic
DEAARRAWEAAERAQ

SEQ ID NO: 37 - protein, 15 aa, Artificial Sequence
  Synthetic
DEEARRVYEAAERAQ

SEQ ID NO: 38 - protein, 15 aa, Artificial Sequence
  Synthetic
DEAARRVYEIVERAQ

SEQ ID NO: 39 - protein, 15 aa, Artificial Sequence
  Synthetic
DEDARRVWELAQKAQ

SEQ ID NO: 40 - protein, 5 aa, Artificial Sequence
  Synthetic
AVVVV

SEQ ID NO: 41 - protein, 5 aa, Artificial Sequence
  Synthetic
AVVIV

SEQ ID NO: 42 - protein, 5 aa, Artificial Sequence
  Synthetic
VVVIV

SEQ ID NO: 43 - protein, 5 aa, Artificial Sequence
  Synthetic
VVLVV

SEQ ID NO: 44 - protein, 5 aa, Artificial Sequence
  Synthetic
AVVFV

SEQ ID NO: 45 - protein, 5 aa, Artificial Sequence
  Synthetic
IVVVV

SEQ ID NO: 46 - protein, 5 aa, Artificial Sequence
  Synthetic
DVILF

SEQ ID NO: 47 - protein, 5 aa, Artificial Sequence
  Synthetic
KVYVF

SEQ ID NO: 48 - protein, 5 aa, Artificial Sequence
  Synthetic
QVIIY

SEQ ID NO: 49 - protein, 5 aa, Artificial Sequence
  Synthetic
VIVIF

SEQ ID NO: 50 - protein, 5 aa, Artificial Sequence
  Synthetic
LVIVY

SEQ ID NO: 51 - protein, 5 aa, Artificial Sequence
  Synthetic
RVVIY

SEQ ID NO: 52 - protein, 5 aa, Artificial Sequence
  Synthetic
IIFVH

SEQ ID NO: 53 - protein, 5 aa, Artificial Sequence
  Synthetic
HIVIV

SEQ ID NO: 54 - protein, 5 aa, Artificial Sequence
  Synthetic
IVVIK

SEQ ID NO: 55 - protein, 5 aa, Artificial Sequence
  Synthetic
LVVIK

SEQ ID NO: 56 - protein, 5 aa, Artificial Sequence
  Synthetic
FVFVH

SEQ ID NO: 57 - protein, 5 aa, Artificial Sequence
  Synthetic
VVIRV

SEQ ID NO: 58 - protein, 5 aa, Artificial Sequence
  Synthetic
FVFVY

SEQ ID NO: 59 - protein, 5 aa, Artificial Sequence
  Synthetic
FVVIF

SEQ ID NO: 60 - protein, 5 aa, Artificial Sequence
  Synthetic
LVFFH

SEQ ID NO: 61 - protein, 5 aa, Artificial Sequence
  Synthetic
FVVFV

SEQ ID NO: 62 - protein, 5 aa, Artificial Sequence
  Synthetic
VVFFR

SEQ ID NO: 63 - protein, 5 aa, Artificial Sequence
  Synthetic
  MISC_FEATURE (1)..(1): X is A, V, or I
  MISC_FEATURE (3)..(3): X is V or L
  MISC_FEATURE (4)..(4): X is V, I or F
XVXXV

SEQ ID NO: 64 - protein, 5 aa, Artificial Sequence
  Synthetic
  MISC_FEATURE (1)..(1): X is D, K, Q, V, L, R, I, or H
  MISC_FEATURE (2)..(2): X is V or I
  MISC_FEATURE (3)..(3): X is I, Y, V, or F
  MISC_FEATURE (4)..(4): X is I, V, or L
  MISC_FEATURE (5)..(5): X is F, Y, H, or V
XXXXX

SEQ ID NO: 65 - protein, 5 aa, Artificial Sequence
  Synthetic
  MISC_FEATURE (1)..(1): X is I, V, L, or F
  MISC_FEATURE (3)..(3): X is I, V, or F
  MISC_FEATURE (4)..(4): X is I, V, R or F
  MISC_FEATURE (5)..(5): X is K, H, V, Y, F, or R
XVXXX

SEQ ID NO: 66 - protein, 88 aa, Artificial Sequence
  Synthetic
DEEEIQKAIEELLRKGVSEEEAAIIIAQRFNVAVVVVVQDERQAKHISEYIRRYIPEADVILFANIVVIKVETEELRKRVWEAAQKAY

SEQ ID NO: 67 - protein, 88 aa, Artificial Sequence
  Synthetic
DEEEIQKAIEELLRKGVSEEEAAIIIVQRFNVAVVVVVQDERQAKHISEYIRRYIPEADVILFANIVVIKVETHELRKRVWEAAQKAY

SEQ ID NO: 68 - protein, 88 aa, Artificial Sequence
  Synthetic
DEEEIQKAIEELLRKGVSEEEAAIIIVQRFNVAVVVVVQDERQAKHISEYIRRYIPEADVILFANLVVIKVETHELRKRVWEAAQKAY

SEQ ID NO: 69 - protein, 88 aa, Artificial Sequence
  Synthetic
DEEEIQKAIEELLRKGVSEEEAAIIIVQTFNVAVVVVVQDERQAKHISEYIRRYIPEADVILFANLVVIKVETDELNKRVWEAAQKAY

SEQ ID NO: 70 - protein, 88 aa, Artificial Sequence
  Synthetic
DEEEIQKAIEELLRKGVSEEEAAIIIVQRFNVAVVVVVQDERQGKHISEYIRRYIPEADVILFANLVVIKVETHELRKRVWEAAQKAY

SEQ ID NO: 71 - protein, 88 aa, Artificial Sequence
  Synthetic
DEEEIQKAIEELLRKGVSEEEAAIIIVQRFNVAVVVVVQDERQGKHISEYIRRYIPEADVILFANLVVIKVETHELSTRVWEAAQKAY

SEQ ID NO: 72 - protein, 88 aa, Artificial Sequence
  Synthetic
DEEEIQKAIEELLRKGVSEEEAAIIIVQRFNVAVVVVVQDERQGKHISEYIRRYIPEADVILFANLVVIKVETHELSQRVWEAAQKAY

SEQ ID NO: 73 - protein, 94 aa, Artificial Sequence
  Synthetic
TEDEEYEERVREALEEAKKKNEHNEELVAQIALLILGAVVVIVAESEEAAKRAAQYVRERVPNAKVYVFDNFVFVHAESREDQEAAWRAAQEAM

SEQ ID NO: 74 - protein, 91 aa, Artificial Sequence
  Synthetic
DEEEEFKRRVQEYQEKYKNENDERDAEAALRAFNAVVVIVVRSEEEAERIAQEIRDRVPEAQVIIYNNLVVIRVDSREAAERVWKIAQEAL

SEQ ID NO: 75 - protein, 93 aa, Artificial Sequence
  Synthetic
DKEDELRELAKELVREYEKRGVSEEEIALLVALAANFAVVIVFDNEEQAERAAKYIKKKLPSAVIVIFNNFVFVYVDSREDERIAWELAQEAQ

SEQ ID NO: 76 - protein, 93 aa, Artificial Sequence
  Synthetic
TEEERLREEVKRVAQRAEEEGANEEEIAQILLLALNAAVVVVVESEEQAERVAQYIREVVPEALVIVYNNFVVIFVESDEAARRAWEAAERAQ

SEQ ID NO: 77 - protein, 93 aa, Artificial Sequence
  Synthetic
DLEERVRKAVNEAQEEAKRNNLDENEAALEAALRLGVVVLVVVDSEEQAERVAKYVHKLVPSVRVVIYNNLVFFHVDTDEEARRVYEAAERAQ

SEQ ID NO: 78 - protein, 94 aa, Artificial Sequence
  Synthetic
DEREEEQRRRLEEVKEEAKRRERSEQDLAVLYLEAVNAAVVFVADSEEEAKRVADIVKKLVPEVIIFVHDNFVVFVVDSDEAARRVYEIVERAQ

SEQ ID NO: 79 - protein, 93 aa, Artificial Sequence
  Synthetic
DEEEEEKEEIRRQLDEARERGVSEEEAAIEVAQNFNFIVVVVVDSEEEAERVAEYVRREVPRVHIVIVQNVVFFRVDSDEDARRVWELAQKAQ

SEQ ID NO: 80 - protein, 88 aa, Artificial Sequence
  Synthetic
EKAAIIAAILVLLLAGVSEEEAAIIIVQAFNVAVVVVVQDERQGKHISEYIRRYIPEAIVILAANLVVIKVETHELSTRVWEAAQKAY

SEQ ID NO: 81 - protein, 88 aa, Artificial Sequence
  Synthetic
DEEEIQKAIEELLRKGVSEEEAAIIIVQRFNVAVVVVVQDEEQGKVISKLIRSLIPEADVILFANLVVIKVETHELSTLVWAAAQAAL

SEQ ID NO: 82 - protein, 88 aa, Artificial Sequence
  Synthetic
DEEEIQKAIEELLRKGVSEEEAAIIIVQRFNVAVVVVVQSERLGKVISEAIRRAIPEADVILFANLVVIKVETHELSTLVWRVAQVLL

SEQ ID NO: 83 - protein, 88 aa, Artificial Sequence
  Synthetic
DEEEIQKAIEELLRKGVSEEEAAIIIVQRFNVAVVVVVQDERQGKAISEFIRRLIPEADVILFANLVVIKVETHELSTRVWRAAQIAF

SEQ ID NO: 84 - protein, 88 aa, Artificial Sequence
  Synthetic
DEEEIQKAIEELLRKGVSEEEAAIIIVQRFNVAVVVVVQDEREGKEISELIRRAIPEADVILFANLVVIKVETHELSTIVWLVAQVAL

SEQ ID NO: 85 - protein, 88 aa, Artificial Sequence
  Synthetic
DEEEIQKAIEELLRKGVSEEEAAIIIVQRFNVAVVVVVQDEREGKEISEEIRKAIPDADVILFANLVVIKVETHELSTVVWLIAQGLM

SEQ ID NO: 86 - protein, 15 aa, Artificial Sequence
  Synthetic
EKAAIIAAILVLLLA

SEQ ID NO: 87 - protein, 12 aa, Artificial Sequence
  Synthetic
EEEAAIIIVQAF

SEQ ID NO: 88 - protein, 14 aa, Artificial Sequence
  Synthetic
EEQGKVISKLIRSL

SEQ ID NO: 89 - protein, 14 aa, Artificial Sequence
  Synthetic
ERLGKVISEAIRRA

SEQ ID NO: 90 - protein, 14 aa, Artificial Sequence
  Synthetic
ERQGKAISEFIRRL

SEQ ID NO: 91 - protein, 14 aa, Artificial Sequence
  Synthetic
EREGKEISELIRRA

SEQ ID NO: 92 - protein, 14 aa, Artificial Sequence
  Synthetic
EREGKEISEEIRKA

SEQ ID NO: 93 - protein, 15 aa, Artificial Sequence
  Synthetic
HELSTLVWAAAQAAL

SEQ ID NO: 94 - protein, 15 aa, Artificial Sequence
  Synthetic
HELSTLVWRVAQVLL

SEQ ID NO: 95 - protein, 15 aa, Artificial Sequence
  Synthetic
HELSTRVWRAAQIAF

SEQ ID NO: 96 - protein, 15 aa, Artificial Sequence
  Synthetic
HELSTIVWLVAQVAL

SEQ ID NO: 97 - protein, 15 aa, Artificial Sequence
  Synthetic
HELSTVVWLIAQGLM

SEQ ID NO: 98 - protein, 5 aa, Artificial Sequence
  Synthetic
IVILA

SEQ ID NO: 99 - protein, 150 aa, Artificial Sequence
  Synthetic
TKEDARSTCEKAARKAAESNDECVAKQAAKDCLEVAKQAGMPTKEAARSFCEAAARAAAESNDEEVAKIAAKACLEVAKQAGMPTKEAARSFCEAAARAVAEAKDPEVAKIAAQACIEVGKQAGMPLKELAKSFVEALVRAAEELNDPEA

SEQ ID NO: 100 - protein, 162 aa, Artificial Sequence
  Synthetic
IVLEAAKRAGIDSKEVLELALRLIEEVLENAQREGYDPEEAIRAAAEAFKRVAEAAKRAGITSSEVLELAIRLIKEVVENAQREGYDISEAARAAAEAFKRVAEAAKRAGITSSETLKRAIEEIRKRVEEAQREGNDISEAARQAAEEFRKKAEELKRCGDV

SEQ ID NO: 101 - protein, 144 aa, Artificial Sequence
  Synthetic
LALEITEQLPDTELAREAQELAREVAKTTDPEAFKVVDLALRIVQQLPDTELAREALELAKEAVKSTDSEALKVVYLALRIVQQLPDTELAREALELAKEAVKSTDQEALKSVYEALQRVQDKPNTEEARESLERAKCDVKSTD

SEQ ID NO: 102 - protein, 131 aa, Artificial Sequence
  Synthetic
AAGGDPELLEVGERIVKELEEQGRSPEEALREAAELLERIRRAAGGDSELIEVAVRIVKELEEQGRSASEAAKEAVELIERIRRAAGGDSDRIKKAVELVRELEERGRSASEAARRAVEEIQRSVECDGGN

SEQ ID NO: 103 - protein, 140 aa, Artificial Sequence
  Synthetic
ALRVVKSRPGSNLAKKALEIILRAAEELAKLPNPSSLKFAVEAAEKVVREQPGSNLAKKALEIILRAAEELAKLPDPEALKEAVKAAEKVVREQPGSELAKKALEIIERAAEELKKSPDPEAQKEAKKAEQKVRCERPGS

SEQ ID NO: 104 - protein, 185 aa, Artificial Sequence
  Synthetic
LLLAIKQNPDNDRAVEEAVRVARKLKKLAEELQEKAKKTGDAKLLTLALTLLLFAVKLVELAIKSNPDNDEAVETAVRLARELKKVAEELQERAKKTGDAELLKLALEALEVAVRAVELAIKSNPDNEEAVETAKRLAEELRKVAELLEERAKETGDPELQELAKRAKEVADRARELAKCSNPNN

SEQ ID NO: 105 - protein, 238 aa, Artificial Sequence
  Synthetic
TKEDARSTCEKAARKAAESNDECVAKQAAKDCLEVAKQAGMPTKEAARSFCEAAARAAAESNDEEVAKIAAKACLEVAKQAGMPTKEAARSFCEAAARAVAEAKDPEVAKIAAQACIEVGKQAGMPLKELAKSFVEALVRAAEELNDPEAEKAAIIAAILVLLLAGVSEEEAAIIIVQAFNVAVVVVVQDERQGKHISEYIRRYIPEAIVILAANLVVIKVETHELSTRVWEAAQKAY

SEQ ID NO: 106 - protein, 250 aa, Artificial Sequence
  Synthetic
DEEEIQKAIEELLRKGVSEEEAAIIIVQRFNVAVVVVVQDEEQGKVISKLIRSLIPEADVILFANLVVIKVETHELSTLVWAAAQAALIVLEAAKRAGIDSKEVLELALRLIEEVLENAQREGYDPEEAIRAAAEAFKRVAEAAKRAGITSSEVLELAIRLIKEVVENAQREGYDISEAARAAAEAFKRVAEAAKRAGITSSETLKRAIEEIRKRVEEAQREGNDISEAARQAAEEFRKKAEELKRCGDV

SEQ ID NO: 107 - protein, 232 aa, Artificial Sequence
  Synthetic
DEEEIQKAIEELLRKGVSEEEAAIIIVQRFNVAVVVVVQSERLGKVISEAIRRAIPEADVILFANLVVIKVETHELSTLVWRVAQVLLLALEITEQLPDTELAREAQELAREVAKTTDPEAFKVVDLALRIVQQLPDTELAREALELAKEAVKSTDSEALKVVYLALRIVQQLPDTELAREALELAKEAVKSTDQEALKSVYEALQRVQDKPNTEEARESLERAKCDVKSTD

SEQ ID NO: 108 - protein, 219 aa, Artificial Sequence
  Synthetic
DEEEIQKAIEELLRKGVSEEEAAIIIVQRFNVAVVVVVQDERQGKAISEFIRRLIPEADVILFANLVVIKVETHELSTRVWRAAQIAFAAGGDPELLEVGERIVKELEEQGRSPEEALREAAELLERIRRAAGGDSELIEVAVRIVKELEEQGRSASEAAKEAVELIERIRRAAGGDSDRIKKAVELVRELEERGRSASEAARRAVEEIQRSVECDGGN

SEQ ID NO: 109 - protein, 228 aa, Artificial Sequence
  Synthetic
DEEEIQKAIEELLRKGVSEEEAAIIIVQRFNVAVVVVVQDEREGKEISELIRRAIPEADVILFANLVVIKVETHELSTIVWLVAQVALALRVVKSRPGSNLAKKALEIILRAAEELAKLPNPSSLKFAVEAAEKVVREQPGSNLAKKALEIILRAAEELAKLPDPEALKEAVKAAEKVVREQPGSELAKKALEIIERAAEELKKSPDPEAQKEAKKAEQKVRCERPGS

SEQ ID NO: 110 - protein, 273 aa, Artificial Sequence
  Synthetic
DEEEIQKAIEELLRKGVSEEEAAIIIVQRFNVAVVVVVQDEREGKEISEEIRKAIPDADVILFANLVVIKVETHELSTVVWLIAQGLMLLLAIKQNPDNDRAVEEAVRVARKLKKLAEELQEKAKKTGDAKLLTLALTLLLFAVKLVELAIKSNPDNDEAVETAVRLARELKKVAEELQERAKKTGDAELLKLALEALEVAVRAVELAIKSNPDNEEAVETAKRLAEELRKVAELLEERAKETGDPELQELAKRAKEVADRARELAKCSNPNN

(19) United States
(12) Patent Application Publication
(10) Pub. No.: US 2023/0272047 A1
(43) Pub. Date: Aug. 31, 2023

(54) Transferrin Receptor Binding Protein

(71) Applicant: UNIVERSITY OF WASHINGTON, Seattle, WA (US)

(72) Inventors: Danny Sahtoe, Seattle, WA (US); Lauren MILLER, Seattle, WA (US); Lance Joseph STEWART, Seattle, WA (US); David BAKER, Seattle, WA (US)

(21) Appl. No.: 18/006,936

(22) PCT Filed: Jul. 28, 2021
(86) PCT No.: PCT/US2021/043469

§ 371 Date: Jan. 26, 2023

Related U.S. Application Data
(60) Provisional application No. 63/058,908, filed on Jul. 30, 2020.

(51) Int. Cl. C07K 14/79; C12N 15/62

(52) U.S. Cl. CPC C07K 14/79; C12N 15/62; C07K 2319/75

(57) ABSTRACT

The present disclosure provides transferring receptor binding polypeptides of the general formula H1-H2-E1-H3-E2-E3-H-4, herein H1, H2, H3, and H4 each independently comprise an alpha, helical domain of between 11-20 amino acids in length; E1, E2, and E3 each independently comprise a beta sheet of 5 amino acids in length; and optional amino acid linkers between domains.

### CROSS REFERENCE

This application claims priority to U.S. Provisional Patent Application Ser. No. 63/058,908 filed Jul. 30, 2020, incorporated by reference herein in its entirety

### FEDERAL FUNDING STATEMENT

This invention was made with government support under Grant Nos. P50 AGO05136 and R01 AG063845, awarded by the National Institutes of Health. The government has certain rights in the invention.

### SEQUENCE LISTING STATEMENT

A computer readable form of the Sequence Listing is filed with this application by electronic submission and is incorporated into this application by reference in its entirety. The Sequence Listing is contained in the file created on Jul. 19, 2021 having the file name “20-9775-WO-SeqList_ST25.txt” and is 55 kb in size.

### BACKGROUND

Human Transferrin Receptor (hTfR) transports transferrin, the major carrier of iron in the body, across the blood brain barrier (BBB) via receptor mediated transcytosis. This process can be exploited to deliver therapeutic payloads into the brain parenchyma that would otherwise be blocked by the BBB. Thus, hTfR is an attractive target candidate for the development of BBB traversing vehicles.

### SUMMARY

In one aspect, the disclosure provides transferrin receptor binding polypeptides 30 comprising the general formula H1-H2-E1-H3-E2-E3-H4, wherein

H1, H2, H3, and H4 each independently comprise an alpha helical domain of between 11-20 amino acids in length;

E1, E2, and E3 each independently comprise a beta sheet of 5 amino acids in length; and

optional amino acid linkers between domains:

wherein the polypeptide binds to the transferrin receptor.

In one embodiment. H1 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-8 and 86, or wherein H1 comprises an amino acid sequence at least 60%, 65%, 70%, 750, 80%, 85%, 90%, 91%, 92%, 93%, 94%, 95%, 96%, 97%, 98%, 99%, or 100% identical to the amino acid sequence selected from the group consisting of SEQ ID NO: 1-8. In another embodiment, H2 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 9-18 and 87, wherein H2 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 9-18. In a further embodiment, H3 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 19-27 and 88-92, or wherein H3 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 19-27. In another embodiment, H4 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 28-39 and 93-97, or H4 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 28-39.

In one embodiment, the polypeptide comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence of H1, H2, H3, and H4 domains from a single row selected from rows (a)-(t) of Table 1. In another embodiment, E1 comprises the amino acid sequence of SEQ ID NO: 63, or E1 comprises the amino acid sequence selected from the group consisting of SEQ ID NO: 40-45. In a further embodiment, E2 comprises the amino acid sequence of SEQ ID NO: 64, or E2 comprises the amino acid sequence selected from the group consisting of SEQ ID NO: 46-53 and 98, or E2 comprises the amino acid sequence selected from the group consisting of SEQ ID NO: 46-53. In another embodiment, E3 comprises the amino acid sequence of SEQ ID NO: 65, or E3 comprises the amino acid sequence selected from the group consisting of SEQ ID NO: 54-62. In one embodiment, the E1, E2, and E3 domains comprise an amino acid sequence at least 60%, 70%, 80%, 90%, 95%, or 100% identical to the amino acid sequence of E1, E2, and E3 domains from a single row of selected from rows (a)-(o) of Table 2, wherein amino acid substitutions relative to the reference domain are conservative amino acid substitutions.

In one embodiment, the polypeptide comprises an amino acid sequence at least 50%, 55%, 60%, 65%, 70%, 75%, 80%, 85%, 90%, 91%, 92%, 93%, 94%, 95%, 96%, 97%, 98%, 99%, or 100% to the amino acid sequence selected from the group consisting of SEQ ID NO: 66-85, or selected from the group consisting of SEQ ID NO: 66-79.

The disclosure also provides recombinant nucleic acid encoding the polypeptides of the disclosure; expression vectors comprising the recombinant nucleic acid of the disclosure operatively linked to a promoter; host cells comprising the polypeptides, nucleic acids, and/or expression vectors of the disclosure; pharmaceutical compositions, comprising the polypeptide, the recombinant nucleic acid, the expression vector, or the recombinant host cell of any of the disclosure, and a pharmaceutically acceptable carrier; and methods for using, or a use of the polypeptide, the recombinant nucleic acid, the expression vector, the recombinant host cell, and/or the pharmaceutical composition of the disclosure, for any suitable purpose including but not limited to treating or limiting arenavirus infection; delivery of therapeutics for treating tumors; and fusion to therapeutics such as biologicals (including but not limited to protein, nucleic acid, and antibody therapeutics) to increase serum half-life of the therapeutic.

### DESCRIPTION OF THE FIGURES

FIG. 1. Computational design pipeline. Short beta sheet motifs are aligned against target edgestrands. After alignment docked strands are minimized and matched to edgestrands present in proteins in a scaffold library. The interface of the resulting edge-to-edge docks are subsequently designed.

FIG. 2(A-B). The human transferrin receptor contains an edgestrand suitable for docking. A) The transferrin receptor ectodomain contains an exposed edge strand (box) that is distant from the transferrin binding site (oval box). B) Docking of a full length de novo ferredoxin-like protein leaves a void region in the interface (left). Removing the C-terminal strand improves potential for interface packing interactions.

FIG. 3(A-E). Design of a human Transferrin receptor binding protein. A) Model of first generation TfR binders (TfR ectodomain, binder). B) Model of the 2nd generation TfR binders (TfR ectodomain, binder). C) 2DS25 (gray, negative control: black) binds to hTfR ectodomain in flow cytometry, 100.000 cells were measured. D) Circular Dichroism chemical denaturation experiment of 2DS25. E) Single concentration biolayer interferometry assay (gray: 2DS25, black: 2DS25_KO (W81A/Q85A).

FIG. 4(A-C). Development of hTfR binders. A) Positions on 2DS25 that improve binding (C-alpha atoms as spheres). B) Biolayer interferometry equilibrium binding curves of 2DS25 and optimized variants. C) Equilibrium binding curves 2DS25 and the new designs 3DS2, 3DS10 and 3DS18.

FIG. 5(A-B). A) Design of 2nd generation hTfR binders. The base ferredoxin scaffold was expanded by building helices h1 and h2 in order to increase the interface buried surface area. B) Histograms of computational metrics of the first and second generation hTfR binders.

FIG. 6(A-E). Characterization of 2DS25. A) Designed model of 2DS25 (yellow) bound to hTfR (gray). B) Flow cytometry histogram (PE signal), 2DS25 does not bind to the polyspecificity reagent (PSR) or beta sheet containing proteins IL17 and CTLA4 indicating 2DS25 is specific. C) Elution profile of 2DS25 on a Superdex 75 10/300 increase GL. D) CD temperature melts full wavelength scan (left) and absorbance at 220 nm (right). E) CD spectra of 2DS25 Guanidine-HCl melts.

FIG. 7. 2DS25 variants biolayer interferometry. Raw kinetic traces of 2DS25 variants binding to hTfR in biolayer interferometry experiments fitted to a 1:1 binding model.

FIG. 8(A-B). Biolayer interferometry traces third generation designs. A) Single concentration binding traces of 7 new designs binding to hTfR. B) Raw kinetic traces of design 2DS25, 3DS2, 3DS10 and 3DS18 fitted to a 1:1 binding model.

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2nd Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.).

As used herein, the singular forms “a”, “an” and “the” include plural referents unless the context clearly dictates otherwise.

As used herein, the amino acid residues arc abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

In all embodiments of polypeptides disclosed herein, any N-terminal methionine residues are optional (i.e.: the N-terminal methionine residue may be present or may be absent, and may be included or excluded when determining percent amino acid sequence identity compared to another polypeptide).

All embodiments of any aspect of the disclosure can be used in combination, unless the context clearly dictates otherwise.

Unless the context clearly requires otherwise, throughout the description and the claims, the words ‘comprise’, “comprising”, and the like are to be construed in an inclusive sense as opposed to an exclusive or exhaustive sense; that is to say, in the sense of “including, but not limited to”. Additionally, the words “herein,” “above.” and “below” and words of similar import, when used in this application, shall refer to this application as a whole and not to any particular portions of the application.

In a first aspect, the disclosure provides transfernn receptor binding polypeptides comprising the general formula H1-H2-E1-H3-E2-E3-H4, wherein

H1, H2, H3, and H4 each independently comprise an alpha helical domain of between 11-20 amino acids in length;

E1, E2, and E3 each independently comprise a beta sheet of 5 amino acids in length; and

optional amino acid linkers between domains; wherein the polypeptide binds to the transferrin receptor.

The polypeptides of the disclosure bind to the TfR apical domain, as discussed in the examples that follow, which also serves as the site for the entry of new world arenaviruses into cells. A number of these viruses such as Machupo, Junin, Guanarito and Sabiá viruses cause hemorrhagic fevers with high fatality rates. Hence, the polypeptides of the disclosure maybe used, for example, to block viral entry into cells. Furthermore, TfR is overexpressed in a number of tumors, and thus the polypeptides of the disclosure may be used to target therapeutics to tumors that express TfR. Similarly, since TfR is expressed throughout the body, the disclosed polypeptides may be exploited as a general delivery platform. Still further, TfR continuously cycles between the cell surface and endocytotic vesicles as part of its natural function to deliver serum Tf into cells. Thus, fusion of biologics to the disclosed polypeptides can be used to increase the in vivo lifetime of the biologic.

Polypeptide binding to the transferrin receptor is determined by biolayer interferometry using an octet instrument, as detailed in the examples that follow. In various embodiments that may be combined with any embodiments herein, the polypeptides bind to the transferrin receptor with a binding affinity of at least 3 μm, 1 μm, 500 nm, 250 nm, 100 nm, or 50 nm.

The various helical domains (H1, H2, H3, and H4) are between 11-20 amino acids in length and may be of any amino acid composition so long as the domains are alpha helical.

In various embodiments, the helical domains may be 12-20, 13-20, 14-20, 15-20, 11-19, 11-18, 11-16, 11-15, 11-14, 11-13, 12-19, 12-18, 12-17, 12-16, 12-15, 12-14, 12-13, 13-29, 13-18, 13-17, 13-16, 13-15, or 13-14 amino acids in length.

In one embodiment, the H1alpha helical domain is between 15 and 20 amino acids in length. In another embodiment, H1 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-8 and 86. In a specific embodiment, H1 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 1-8.

>2DS25
(SEQ ID NO: 1)
DEEEIQKAIEELDRK
>2DS25_DHR7N
(SEQ ID NO: 86)
EKAAIIAAILVILLA
>3DS2
(SEQ ID NO: 2)
TEDEEYEERVREALEEAKKK
>3DS4
(SEQ ID NO: 3)
DEEEEFKREVQEYQEKY
>3DS6
(SEQ ID NO: 4)
DKEDELRELAKELVREYEKR
>3DS10
(SEQ ID NO: 5)
TEEERLREEVKRVAQRAEEE
>3DS11
(SEQ ID NO: 6)
DLEERVRKAVNEAQEEAKRN
>3DS18
(SEQ ID NO: 7)
DEREEEQRRRLEEVKEEAKR
>3DS23
(SEQ ID NO: 8)
DEEEEEKEEIRRQLDEARER

As described in the examples that follow, the inventors have conducted extensive mutational and functional analysis of the polypeptides of the disclosure, identifying residues that are involved at the interface when bound to transferring receptor and those that are not, thus providing detailed teaching of how the polypeptides may be modified while retaining transferring receptor binding activity.

In another embodiment, at least 40%, 50%, or 60% of residues in alpha helical domain H2 are hydrophobic. In a further embodiment, H2 is between 11-13 amino acids in length. In various embodiments, H2 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 9-18 and 87. In a further embodiment, H2 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 9-18.

>2DS25
(SEQ ID NO: 9)
EEEAAIIIAQRF
>2DS25.1
(SEQ ID NO: 10)
EEEAAIIIVQRF
>2DS25.3
(SEQ ID NO: 11)
EEEAAIIIVQTF
>2DS25_DHR7N
(SEQ ID NO: 87)
EEEAAIIIVQAF
>3DS2
(SEQ ID NO: 12)
EELVAQIALLIL
>3DS4
(SEQ ID NO: 13)
DERDABAALRA
>3DS6
(SEQ ID NO: 14)
EEEIALLVALAA
>3DS10
(SEQ ID NO: 15)
EEEIAQILLLAL
>3DS11
(SEQ ID NO: 16)
ENEAALEAALRL
>3DS18
(SEQ ID NO: 17)
EQDLAVLYLEAV
>3DS23
(SEQ ID NO: 18)
DEEEAAIEVAQNF

In another embodiment, H2 residues in bold font are conserved relative to the reference amino acid sequence (i.e.: relative to SEQ ID NO: 9-18 and 87). These residues have been shown to participate in transferring receptor binding.

In one embodiment, the H3 alpha helical domain is between 13-14 amino acids in length. In another embodiment, H3 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 19-27 and 88-92. In a further embodiment, H3 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 19-27.

>2DS25
(SEQ ID NO: 19)
ERQAKHISEYIRRY
>2DS25.4
(SEQ ID NO: 20)
ERQGKHISEYIRRY
>2DS25_DHR10C
(SEQ ID NO: 88)
EEQGKVISKLIRSL
>2DS25_DHR21C
(SEQ ID NO: 89)
ERLGKVISEAIRRA
>2DS25_DHR39C
(SEQ ID NO: 90)
ERQGKAISEFIRRL
>2DS25_DHR53C
(SEQ ID NO: 91)
EREGKEISELIRRA
>2DS25_DHR82C
(SEQ ID NO: 92)
EREGKEISEEIRKA
>3DS2
(SEQ ID NO: 21)
EEAAKRAAQYVRER
>3DS4
(SEQ ID NO: 22)
EEAERIAQEIRDR
>3DS6
(SEQ ID NO: 23)
EEQAERAAKYIKKK
>3DS10
(SEQ ID NO: 24)
EEQAERVAQYIREV
>3DS11
(SEQ ID NO: 25)
EEQAERVAKYVHKL
>3DS18
(SEQ ID NO: 26)
EEEAKRVADIVKKL
>3DS23
(SEQ ID NO: 27)
EEEAERVAEYVRRE

In one embodiment, alpha helical domain H4 is between 14-15 amino acids in length. In another embodiment, H4 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 28-39 and 93-97. In a further embodiment, H4 comprises an amino acid sequence at least 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 28-39.

>2DS25
(SEQ ID NO: 28)
EELRKRVWEAAQKAY
>2DS25.1
(SEQ ID NO: 29)
HELRKRVWEAAQKAY
>2DS25.3
(SEQ ID NO: 30)
DELNKRVWEAAQKAY
>2DS25.5
(SEQ ID NO: 31)
HELSTRVWEAAQKAY
>2DS25.6
(SEQ ID NO: 32)
HELSQRVWEAAQKAY
>2DS25_DHR10C
(SEQ ID NO: 93)
HELSTLVWAAAQAAL
>2DS25_DHR21C
(SEQ ID NO: 94)
HELSTLVWRVAQVLL
>2DS25_DHR39C
(SEQ ID NO: 95)
HELSTRVWRAAQIAF
>2DS25_DHR53C
(SEQ ID NO: 96)
HELSTIVWLVAQVAL
>2DS25_DHR82C
(SEQ ID NO: 97)
HELSTVVWLIAQGLM
>3DS2
(SEQ ID NO: 33)
REDQEAAWRAAQEAM
>3DS4
(SEQ ID NO: 34)
EAAERVWKIAQEAL
>3DS6
(SEQ ID NO: 35)
REDERIAWELAQEAQ
>3DS10
(SEQ ID NO: 36)
DEAARRAWEAAERAQ
>3DS11
(SEQ ID NO: 37)
DEEARRVYEAAERAQ
>3DS18
(SEQ ID NO: 38)
DEAARRVYEIVERAQ
>3DS23
(SEQ ID NO: 39)
DEDARRVWELAQKAQ

In one embodiment, bold residues in the H4 domains are conserved relative to the reference polypeptide. These residues have been shown to participate in transferring receptor binding.

In another embodiment, transferrin receptor binding polypeptides of the disclosure comprise H1, H2, H3, and H4 domains that comprise an amino acid sequence at least 60%, 65%, 70%, 75%, 80%, 85%, 90%, 91%, 92%, 93%, 94%, 95%, 96%, 97%, 98%, 99%, or 100% identical to the amino acid sequence of H1, H2, H3, and H4 domains from a single row selected from rows (a)-(t) of Table 1. In another embodiment, transferrin receptor binding polypeptides of the disclosure comprise H1, H2. H3, and H4 domains that comprise an amino acid sequence at least 60%, 65%, 70%, 75%, 80%, 85%, 90%, 91%, 92%, 93%, 94%, 95%, 96%, 97%, 98%, 99%, or 100% identical to the amino acid sequence of H1, H2, H3, and H4 domains from a single row selected from rows (a)-(n) of Table 1. Rows (a)-(g) and (o)-(t) are based on “2D designs as described in more detail in the examples (see naming convention for specific polypeptides and domains, i.e.: “2DS25”, etc.), while rows (h)-(n) are based on “3D designs” (i.e.: 3DS2, 3DS4, etc.).

TABLE 1

| | H1 | H2 | H3 | H4 |
|---|---|---|---|---|
| (a) | DEEEIQKA | EEEAAI | ERQAKHI | EELRKRVW |
| | IEELLRK | IIAQRE | SEYIRRY | EAAQKAY |
| | (SEQ ID | (SEQ ID | (SEQ ID | (SEQ ID |
| | NO: 1) | NO: 9) | NO: 19) | NO: 28) |
| (b) | DEEEIQKA | EEEAAI | ERQAKHI | HELRKRVW |
| | IEELLRK | IIVQRF | SEYIRRY | EAAQKAY |
| | (SEQ ID | (SEQ ID | (SEQ ID | (SEQ ID |
| | NO: 1) | NO: 10) | NO: 19) | NO: 29) |
| (c) | DEEEIQKA | EEEAAI | ERQAKHI | HELRKRVW |
| | IEELLRK | IIVQRF | SEYIRRY | EAAQKAY |
| | (SEQ ID | (SEQ ID | (SEQ ID | (SEQ ID |
| | NO: 1) | NO: 10) | NO: 19) | NO: 29) |
| (d) | DEEEIQKA | EEEAAI | ERQAKHI | DELNKRVW |
| | IEELLRK | IIVQTE | SEYIRRY | EAAQKAY |
| | (SEQ ID | (SEQ ID | (SEQ ID | (SEQ ID |
| | NO: 1) | NO: 11) | NO: 19) | NO: 30) |
| (e) | DEEEIQKA | EEEAAI | ERQGKHI | HELRKRVW |
| | IEELLRK | IIVQRF | SEYIRRY | EAAQKAY |
| | (SEQ ID | (SEQ ID | (SEQ ID | (SEQ ID |
| | NO: 1) | NO: 10) | NO: 20) | NO: 29) |
| (f) | DEEEIQKA | EEEAAI | ERQGKHI | HELSTRVW |
| | IEELLRK | IIVQRF | SEYIRRY | EAAQKAY |
| | (SEQ | (SEQ ID | (SEQ ID | (SEQ ID |
| | NO: 1) | NO: 10) | NO: 20) | NO: 31) |
| (g) | DEEEIQKA | EEEAAI | ERQGKHI | HELSQRVW |
| | IEELLRK | IIVQRF | SEYIRRY | EAAQKAY |
| | (SEQ ID | (SEQ ID | (SEQ ID | (SEQ ID |
| | NO: 1) | NO: 10) | NO: 20) | NO: 32) |
| (h) | TEDEEYEE | EELVAQ | EEAAKRA | REDQEAAW |
| | RVREALEE | IALLIL | AQYVRER | RAAQEAM |
| | AKKK | (SEQ ID | (SEQ ID | (SEQ ID |
| | (SEQ ID | NO: 12) | NO: 21) | NO: 33) |
| | NO: 2) | | | |
| (i) | DEEEEFKR | DERDAE | EEAERIA | EAAERVW |
| | RVQEYQEK | AALRA | QEIRDR | KIAQEAL |
| | Y | (SEQ ID | (SEQ ID | (SEQ ID |
| | (SEQ ID | NO: 13) | NO: 22) | NO: 34) |
| | NO: 3) | | | |
| (j) | DKEDELRE | EEEIAL | EEQAERA | REDERIAW |
| | LAKELVRE | LVALAA | AKYIKKK | ELAQEAQ |
| | YEKR | (SEQ ID | (SEQ ID | (SEQ ID |
| | (SEQ ID | NO: 14) | NO: 23) | NO: 35) |
| | NO: 4) | | | |
| (k) | TEEERLRE | EEEIAQ | EEQAERV | DEAARRAW |
| | EVKRVAQR | ILLLAL | AQYIREV | EAAERAQ |
| | AEEE | (SEQ ID | (SEQ ID | (SEQ ID |
| | (SEQ ID | NO: 15) | NO: 24) | NO: 36) |
| | NO: 5) | | | |
| (l) | DLEERVRK | ENEAAL | EEQAERV | DEEARRVY |
| | AVNEAQEE | EAALRL | AKYVHKL | EAAERAQ |
| | AKRN | (SEQ ID | (SEQ ID | (SEQ ID |
| | (SEQ ID | NO: 16) | NO: 25) | NO: 37) |
| | NO: 6) | | | |
| (m) | DEREEEQR | EQDLAV | EEEAKRV | DEAARRVY |
| | RRLEEVKE | LYLEAV | ADIVKKL | EIVERAQ |
| | EAKR | (SEQ ID | (SEQ ID | (SEQ ID |
| | (SEQ ID | NO: 17) | NO: 26) | NO: 38) |
| | NO: 7) | | | |
| (n) | DEEEEEKE | DEEEAAI | EEEAERV | DEDARRVW |
| | EIRRQLDE | EVAQNF | AEYVRRE | ELAQKAQ |
| | ARER | (SEQ ID | (SEQ ID | (SEQ ID |
| | (SEQ ID | NO: 18) | NO: 27) | NO: 39) |
| | NO: 8) | | | |
| (o) | EKAAIIAA | EEEAAII | ERQGKHI | HELSTRVW |
| | ILVILLA | IVQAF | SEYIRRY | EAAQKAY |
| | (SEQ ID | (SEQ ID | (SEQ ID | (SEQ ID |
| | NO: 86) | NO: 87) | NO: 20) | NO: 31) |
| (p) | DEEEIQKA | EEEAAII | EEQGKVI | HELSTLVW |
| | IEELLRK | IVQRF | SKLIRSL | AAAQAAL |
| | (SEQ ID | (SEQ ID | (SEQ ID | (SEQ ID |
| | NO: 1) | NO: 10) | NO: 88) | NO: 93) |
| (q) | DEEEIQKA | EEEAAII | ERLGKVI | HELSTLVW |
| | IEELLRK | IVQRF | SEAIRRA | RVAQVLL |
| | (SEQ ID | (SEQ ID | (SEQ ID | (SEQ ID |
| | NO: 1) | NO: 10) | NO: 89) | NO: 94) |
| (r) | DEEEIQKA | EEEAAII | ERQGKAI | HELSTRVW |
| | IEELLRK | IVQRF | SEFIRRL | RAAQIAF |
| | (SEQ ID | (SEQ ID | (SEQ ID | (SEQ ID |
| | NO: 1) | NO: 10) | NO: 90) | NO: 95) |
| (s) | DEEEIQKA | EEEAAII | EREGKEI | HELSTIVW |
| | IEELLRK | IVQRF | SELIRRA | LVAQVAL |
| | (SEQ ID | (SEQ ID | (SEQ ID | (SEQ ID |
| | NO: 1) | NO: 10) | NO: 91) | NO: 96) |
| (t) | DEEEIQKA | EEEAAII | EREGKEI | HELSTVVW |
| | IEELLRK | IVQRF | SEEIRKA | LIAQGLM |
| | (SEQ ID | (SEQ ID | (SEQ ID | (SEQ ID |
| | NO: 1) | NO: 10) | NO: 92) | NO: 97) |

The transferrin receptor binding polypeptides of the disclosure comprise E1, E2, and E3 domains that independently comprise a beta sheet of 5 amino acids in length. In one embodiment, at least 3, 4, or all 5 of the amino acids in each of the E1, E2, and E3 domains are hydrophobic.

In another embodiment, the E1 domain comprises the amino acid sequence (A/V/I)V(V/L)(V/I/F)V (SEQ ID NO:63), wherein residues in parentheses are alternative residues at a given position. In a further embodiment, the E1 domain comprises the amino acid sequence selected from the group consisting of SEQ ID NO: 40-45.

(SEQ ID NO: 40)
AVVVV
(SEQ ID NO: 41)
AVVIV
(SEQ ID NO: 42)
VVVIV
(SEQ ID NO: 43)
VVLVV
(SEQ ID NO: 44)
AVVFV
(SEQ ID NO: 45)
IVVVV

In a further embodiment, the E2 domain comprises the amino acid sequence (D/K/Q/V/L/R/I/H)(V/I)(I/Y/V/F)(L/V/I)(F/Y/H/V) (SEQ ID NO:64). In a still further embodiment, E2 comprises the amino acid sequence selected from the group consisting of SEQ ID NO: 46-53 and 98, or wherein E2 comprises the amino acid sequence selected from the group consisting of SEQ ID NO: 46-53.

(SEQ ID NO: 46)
DVILF
(SEQ ID NO: 47)
KVYVF
(SEQ ID NO: 48)
QVIIY
(SEQ ID NO: 49)
VIVIF
(SEQ ID NO: 50)
LVIVY
(SEQ ID NO: 51)
RVVIY
(SEQ ID NO: 52)
IIFVH
(SEQ ID NO: 53)
HIVIV
(SEQ ID NO: 98)
IVILA

In one embodiment, the E3 domain comprises the amino acid sequence (I/V/L/F)V(V/F/1)(I/V/R/F/)(K/H/V/Y/F/R) (SEQ ID NO:65). In other embodiments, E3 comprises the amino acid sequence selected from the group consisting of SEQ ID NO: 54-62.

(SEQ ID NO: 54)
IVVIK
(SEQ ID NO: 55)
LVVIK
(SEQ ID NO: 56)
FVFVH
(SEQ ID NO: 57)
VVIRV
(SEQ ID NO: 58)
FVFVY
(SEQ ID NO: 59)
FVVIF
(SEQ ID NO: 60)
LVFFH
(SEQ ID NO: 61)
FVVFV
(SEQ ID NO: 62)
VVFFR

In a further embodiment, the transferrin receptor binding polypeptide comprises E1, E2, and E3 domains that comprise an amino acid sequence at least 60%, 70%, 80%, 90%, 95%, or 100% identical to the amino acid sequence of E1, E2, and E3 domains from a single row of selected from rows (a)-(o) of Table 2, wherein amino acid substitutions relative to the reference domain are conservative amino acid substitutions. In another embodiment the transferrin receptor binding polypeptide comprises E1, E2, and E3 domains that comprise an amino acid sequence at least 60%, 70%, 80%, 90%, 95%, or 100% identical to the amino acid sequence of E1. E2, and E3 domains from a single row of selected from rows (a)-(n) of Table 2, wherein amino acid substitutions relative to the reference domain are conservative amino acid substitutions. Rows (a)-(g) and (o) are based on “2D designs as described in more detail in the examples, while rows (h)-(n) arc based on “3D designs”.

| | E1 | E2 | E3 |
|---|---|---|---|
| (a) | AVVVV (SEQ ID | DVILF (SEQ ID | IVVIK (SEQ ID |
| | NO: 40) | NO: 46) | NO: 54) |
| (b) | AVVVV (SEQ ID | DVILF (SEQ ID | IVVIK (SEQ ID |
| | NO: 40) | NO: 46) | NO: 541 |
| (c) | AVVVV (SEQ ID | DVILF (SEQ ID | LVVIK (SEQ ID |
| | NO: 40) | NO: 46) | NO: 55) |
| (d) | AVVVV (SEQ ID | DVILF (SEQ ID | LVVIK (SEQ ID |
| | NO: 40) | NO: 46) | NO: 55) |
| (e) | AVVVV (SEQ ID | DVILF (SEQ ID | LVVIK (SEQ ID |
| | NO: 40) | NO: 46) | NO: 55) |
| (f) | AVVVV (SEQ ID | DVILF (SEQ ID | LVVIK (SEQ ID |
| | NO: 40) | NO: 46) | NO: 55) |
| (g) | AVVVV (SEQ ID | DVILF (SEQ ID | LVVIK (SEQ ID |
| | NO: 40) | NO: 46) | NO: 55) |
| (h) | VVVIV (SEQ ID | KVYVF (SEQ ID | FVFVH (SEQ ID |
| | NO: 41) | NO: 47) | NO: 56) |
| (i) | VVVIV (SEQ ID | QVIIY (SEQ ID | VVIRV (SEQ ID |
| | NO: 41) | NO: 48) | NO: 57) |
| (j) | AVVIV (SEQ ID | VIVIF (SEQ ID | FVFVY (SEQ ID |
| | NO: 42) | NO: 49) | NO: 58) |
| (k) | AVVVV (SEQ ID | LVIVY (SEQ ID | FVVIF (SEQ ID |
| | NO: 40) | NO: 50) | NO: 59) |
| (l) | VVLVV (SEQ ID | RVVIY (SEQ ID | LVFFH (SEQ ID |
| | NO: 43) | NO: 51) | NO: 60) |
| (m) | AVVFV (SEQ ID | IIFVH (SEQ ID | FVVFV (SEQ ID |
| | NO: 44) | NO: 52 ) | NO: 61) |
| (n) | IVVVV (SEQ ID | HIVIV (SEQ ID | VVFFR (SEQ ID |
| | NO: 45) | NO: 53) | NO: 62) |
| (o) | AVVVV (SEQ ID | DVILF (SEQ ID | IVILA (SEQ ID |
| | NO: 40) | NO: 46) | NO: 98) |

The transferrin receptor binding polypeptides of the disclosure may comprise amino acid linkers between one or more adjacent domains. When such amino acid linker(s) are present, they may be present between only 2 adjacent domains (for example, an amino acid linker between H1 and H2 domains, and no linkers present between other domains), between multiple adjacent domains, or between all adjacent domains. The amino acid linker may be of any suitable length and amino acid composition. In one embodiment, amino acid linkers, when present, are independently between 2-4 amino acids in length.

In other embodiments, the transferrin receptor binding polypeptide comprises an amino acid sequence at least 501%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 66-85, or selected from the group consisting of SE ID NO: 66-79.

>2DS25
(SEQ ID NO: 66)
DEEEIQKAIEELLRKGVSEEEAAIIIAQRFNVAVVVVVQDERQA
KHISEYIRRYIPEADVILFANIVVIKVETEELRKRVWEAAQKAY
>2DS25.1
(SEQ ID NO: 67)
DEEEIQKAIEELLRKGVSEEEAAIIIVQRFNVAVVVVVQDERQA
KHISEYIRRYIPEADVILFANIVVIKVETHELRKRVWEAAQKAY
>2DS25.2
(SEQ ID NO: 68)
DEEEIQKAIEELLRKGVSEEEAAIIIVQRFNVAVVVVVQDERQA
KHISEYIRRYIPEADVILFANLVVIKVETHELRKRVWEAAQKAY
>2DS25.3
(SEQ ID NO: 69)
DEEEIQKAIEELLRKGVSEEEAAIIIVQTFNVAVVVVVQDERQA
KHISEYIRRYIPEADVILFANLVVIKVETDELNKRVWEAAQKAY
>2DS25.4
(SEQ ID NO: 70)
DEEEIQKAIEELLRKGVSEEEAAIIIVQRFNVAVVVVVQDERQG
KHISEYIRRYIPEADVILFANLVVIKVETHELRKRVWEAAQKAY
>2DS25.5
(SEQ ID NO: 71)
DEEEIQKAIEELLRKGVSEEEAAIIIVQRFNVAVVVVVQDERQG
KHISEYIRRYIPEADVILFANLVVIKVETHELSTRVWEAAQKAY
>2DS25.6
(SEQ ID NO: 72)
DEEEIQKAIEELLRKGVSEEEAAIIIVQRFNVAVVVVVQDERQG
LKHISEYIRRYIPEADVILFANLVVIKVETHESQRVWEAAQKAY
>3DS2
(SEQ ID NO: 73)
TEDEEYEERVREALEEAKKKNEHNEELVAQIALLILGAVVVIVA
ESEEAAKRAAQYVRERVPNAKVYVFDNFVFVHAESREDQEAAWR
AAQEAM
>3DS4
(SEQ ID NO: 74)
DEEEEFKRRVQEYQEKYKNENDERDAEAALRAFNAVVVIVVRSE
DEEAERIAQEIRDRVPEAQVIIYNNLVVIRVSREAAERVWKIAQ
EAL
>3DS6
(SEQ ID NO: 75)
DKEDELRELAKELVREYEKRGVSEEEIALLVALAANFAVVIVFD
NEEQAERAAKYIKKKLPSAVIVIFNNFVFVYVDSREDERIAWEL
AQEAQ
>3DS10
(SEQ ID NO: 76)
TEEERLREEVKRVAQRAEEEGANEEEIAQILLLALNAAVVVVVE
SEEQAERVAQYIREVVPEALVIVYNNFVVIFVESDEAARRAWEA
AERAQ
>3DS11
(SEQ ID NO: 77)
DLEERVRKAVNEAQEEAKRNNLDENEAALEAALRLGVVVLVVVD
SEEQAERVAKYVHKLVPSVRVVIYNNLVFFHVDTDEEARRVYEA
AERAQ
>3DS18
(SEQ ID NO: 78)
DEREEEQRRRLEEVKEEAKRRERSEQDLAVLYLEAVNAAVVFVA
DSEEEAKRVADIVKKLVPEVIIFVHDNFVVFVVDSDEAARRVYE
IVERAQ
>3DS23
(SEQ ID NO: 79)
DEEEEEKEEIRRQLDEARERGVSEEEAAIEVAQNFNFIVVVVVD
SEEEAERVAEYVRREVPRVHIVIVQNVVFFRVDSDEDARRVWEL
AQKAQ
>2DS25_DHR7N
(SEQ ID NO: 80)
EKAAIIAAILVLLLAGVSEEEAAIIIVQAFNVAVVVVVQDERQG
KHISEYIRRYIPEAIVILAANLVVIKVETHELSTRVWEAAQKAY
>2DS25_DHR10C
(SEQ ID NO: 81)
DEEEIQKAIEELLRKGVSEEEAAIIIVQRFNVAVVVVVQDEEQG
KVISKLIRSLIPEADVILFANLVVIKVETHELSTLVWAAAQAAL
>2DS25_DHR21C
(SEQ ID NO: 82)
DEEEIQKAIEELLRKGVSEEEAAIIIVQRFNVAVVVVVQSERLG
KVISEAIRRAIPEADVILFANLVVIKVETHELSTLVWRVAQVLL
>2DS25_DHR39C
(SEQ ID NO: 83)
DEEEIQKAIEELLRKGVSEEEAAIIIVQRFNVAVVVVVQDERQG
KAISEFIRRLIPEADVILFANLVVIKVETHELSTRVWRAAQIAF
>2DS25_DHR53C
(SEQ ID NO: 84)
DEEEIQKAIEELLRKGVSEEEAAIIIVQRFNVAVVVVVQDEREG
KEISELIRRAIPEADVILFANLVVIKVETHELSTIVWLVAQVAL
>2DS25_DHR82C
(SEQ ID NO: 85)
DEEEIQKAIEELLRKGVSEEEAAIIIVQRFNVAVVVVVQDEREG
KEISEEIRKAIPDADVILFANLVVIKVETHELSTVVWLIAQGLM

In one embodiment, amino acid substitutions relative to the reference polypeptide are at surface residues that are not in or near the interface. Table 3 lists the residue positions that are surface residues that are not in or near the interface. As will be understood by those of skill in the art, these residues are not present at or near a binding interface of the polypeptides of the disclosure and transferrin receptor (as detailed in the examples), and thus are more readily mutable without impacting transferring receptor binding activity.

TABLE 3
All surface residues per design that are NOT in or near interface:

All 2D designs: [1, 2, 3, 4, 7, 8, 10, 11, 13, 14, 15, 16, 17, 40, 41, 42,
43, 45, 46, 49, 52, 53, 54, 56, 57, 59, 60, 62, 64, 72]
3DS2: [1, 2, 3, 4, 5, 6, 7, 8, 9, 11, 12, 15, 16, 19, 20, 22, 23, 35, 46,
47, 48, 51, 52, 55, 58, 59, 60, 62, 63, 65, 66, 68, 70, 78, 79]
3DS4: [1, 2, 3, 4, 5, 7, 8, 9, 11, 12, 13, 14, 15, 16, 17, 18, 19, 20, 43,
44, 45, 48, 49, 52, 53, 56, 57, 59, 60, 62, 63, 65, 67, 75, 76]
3DS6: [1, 2, 3, 4, 5, 8, 11, 12, 13, 15, 16, 18, 19, 20, 21, 22, 45, 46,
47, 50, 51, 54, 57, 58, 59, 61, 62, 65, 67, 69, 77, 78]
3DS10: [1, 2, 3, 4, 5, 7, 8, 9, 11, 12, 15, 16, 18, 19, 20, 21, 22, 34,
45, 46, 47, 50, 51, 54, 58, 59, 61, 62, 65, 67, 69, 77, 78]
3DS11: [1, 3, 4, 5, 7, 8, 11, 12, 14, 15, 16, 18, 19, 20, 21, 22, 45, 46,
47, 50, 51, 54, 58, 59, 61, 62, 64, 65, 67, 69, 77, 78, 80]
3DS18: [1, 2, 3, 4, 5, 6, 8, 9, 12, 13, 15, 16, 17, 19, 20, 21, 22, 23,
35, 46, 47, 48, 51, 52, 55, 59, 60, 62, 63, 65, 66, 68, 69, 70, 78, 79]
3DS23: [1, 2, 3, 4, 5, 6, 8, 9, 11, 12, 13, 15, 16, 18, 19, 20, 21,
45, 46, 47, 50, 51, 54, 55, 58, 59, 61, 62, 64, 65, 67, 69, 77, 78]

The transferrin receptor binding polypeptides of the disclosure may comprise additional residues. In some embodiments, the polypeptides may comprise additional residues at the N-terminus and/or C-terminus of the polypeptides. Any additional residues may be added as deemed appropriate for an intended purpose. In various non-limiting embodiments, the polypeptide may further comprise a functional domain. The polypeptides may comprise any additional functional domain(s), including but not limited to detection domains, stabilization domains, therapeutic moieties, diagnostic moieties and drug delivery vehicle. The functional domains may be added as a translational fusion with the polypeptide, or may be chemically coupled to the polypeptide. Any suitable chemical coupling may be used, including but not limited to covalent linkage to a cysteine residue. For example any surface amino acid residue in the polypeptide not present at or near the binding interface (see Table 3) can be mutated to cysteine. In one embodiment, the one or more additional functional domains are present at the N and/or C terminus of the polypeptide as a translational fusion. In one embodiment, the one or more functional domains comprises a stabilization domain, including but not limited to polyethylene glycol (PEG), albumin, hydroxyethyl starch (HES), conformationally disordered polypeptide sequence composed of the amino acids Pro, Ala, and/or Ser (‘PASylation’), and/or a mucin diffusivity polypeptide composed of amino acids Lys and Ala, with or without Glu.

In another embodiment, the functional domain may comprise a helical repeat protein. This embodiment results in a polypeptide with a longer residency time in the blood. In non-limiting embodiments, the helical repeat proteins comprises an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 99-104.

>DHR7N
(SEQ ID NO: 99)
TKEDARSTCEKAARKAAESNDECVAKQAAKDCLEVAKQAGMPTKEAARSF
CEAAARAAAESNDEEVAKIAAKACLEVAKQAGMPTKEAARSFCEAAARAV
AEAKDPEVAKIAAQACIEVGKQAGMPLKELAKSFVEALVRAAEELNDPEA
>DHR10C
(SEQ ID NO: 100)
IVLEAAKRAGIDSKEVLELALRLIEEVLENAQREGYDPEEAIRAAAEAFK
RVAEAAKRAGITSSEVLELAIRLIKEVVENAQREGYDISEAARAAAEAFK
RVAEAAKRAGITSSETLKRAIEEIRKRVEEAQREGNDISEAARQAAEEFR
KKAEELKRCGDV
>DHR21C
(SEQ ID NO: 101)
LALEITEQLPDTELAREAQELAREVARTTDPEAFKVVDLALRIVQQLPDT
ELAREALELAKEAVKSTDSEALKVVYLALRIVQQLPDTELAREALELAKE
AVKSTDQEALKSVYEALQRVQDKPNTEEARESLERAKCDVKSTD
>DHR39C
(SEQ ID NO: 102)
AAGGDPELLEVGERIVKELEEQGRSPEEALREAAELLERIRRAAGGDSEL
IEVAVRIVKELEEQGRSASEAAKEAVELIERIRRAAGGDSDRIKKAVELV
RELEERGRSASEAARRAVEEIQRSVECDGGN
>DHR53C
(SEQ ID NO: 103)
ALRVVKSRPGSNLAKKALEIILRAAEELAKLPNPSSLKFAVEAAEKVVRE
QPGSNLAKKALEIILRAAEELAKLPDPEALKEAVKAAEKVVREQPGSELA
KKALEIIERAAEELKKSPDPEAQKEAKKAEQKVRCERPGS
>DHR82C
((SEQ ID NO: 104)
LLLAIKQNPDNDRAVEEAVRVARKLKKLAEELQEKAKKTGDAKLLTLALT
LLLFAVKLVELAIKSNPDNDEAVETAVRLARELKKVAEELQERAKKTGDA
ELLKLALEALEVAVRAVELAIKSNPDNEEAVETAKRLAEELRKVAELLEE
RAKETGDPELQELAKRAKEVADRARELAKCSNPNN

In exemplary embodiments, polypeptides of this embodiment comprise an amino acid sequence at least 50%, 55%, 60%, 65%, 70%, 75%, 80%, 85%, 90%, 91%, 92%, 93%, 94%, 95%, 96%, 97%, 98%, 99%, or 100% identical to the amino acid sequence selected from the group consisting of SEQ ID NO: 105-110.

>2DS25_DHR7N
(SEQ ID NO: 105)
TKEDARSTCEKAARKAAESNDECVAKQAAKDCLEVAKQAGMPTKEAARSF
CEAAARAAAESNDEEVAKIAAKACLEVAKQAGMPTKEAARSFCEAAARAV
AEAKDPEVAKIAAQACIEVGKQAGMPLKELAKSFVEALVRAAEELNDPEA
EKAAIIAAILVLLLAGVSEEEAAIIIVQA
FNVAVVVVVQDERQGKHISEYIRRYIPEA
IVILAANLVVIKVETHELSTRVWEAAQKAY
>2DS25_DHR10C
(SEQ ID NO: 106)
DEEEIQKAIEELLEKGVSEEEAAIIIVQR
FNVAVVVVVQDEEQGKVISKLIRSLIPEA
DVILFANLVVIKVETHELSTLVWAAAQAAL
IVLEAAKRAGIDSKEVLELALRLIEEVLENAQREGYDPEEAIRAAAEAFK
RVAEAAKRAGITSSEVLELAIRLIKEVVENAQREGYDISEAARAAAEAFK
RVAEAAKRAGITSSETLKRAIEEIRKRVEEAQREGNDISEAARQAAEEFR
KKAEELKRCGDV
>2DS25_DHR21C
(SEQ ID NO: 107)
DEEEIQKAIEELLRKGVSEEEAAIIIVQR
FNVAVVVVVQSERLGKVISEAIRRAIPEA
DVILFANLVVIKVETHELSTLVWRVAQVLL
LALEITEQLPDTELAREAQELAREVAKTTDPEAFKVVDLALRIVQQLPDT
ELAREALELAKEAVKSTDSEALKVVYLALRIVQQLPDTELAREALELAKE
AVKSTDQEALKSVYEALQRVQDKPNTEEARESLERAKCDVKSTD
>2DS25_DHR39C
(SEQ ID NO: 108)
DEEEIQKAIEELLRKGVSEEEAAIIIVQR
FNVAVVVVVQDERQGKAISEFIRRLIPEA
DVILFANLVVIKVETHELSTRVWRAAQIAF
AAGGDPELLEVGERIVKELEEQGRSPEEALREAAELLERIRRAAGGDSEL
IEVAVRIVKELEEQGRSASEAAKEAVELIERIRRAAGGDSDRIKKAVELV
RELEERGRSASEAARRAVEEIQRSVECDGGN
>2DS25_DHR53C
(SEQ ID NO: 109)
DEEEIQKAIEELLEKGVSEEEAAIIIVQR
FNVAVVVVVQDEREGKEISELIRRAIPEA
DVILFANLVVIKVETHELSTIVWLVAQVAL
ALRVVKSRPGSNLAKKALEIILRAAEELAKLPNPSSLKFAVEAAEKVVRE
QPGSNLAKKALEIILRAAEELAKLPDPEALKEAVKAAEKVVREQPGSELA
KKALEIIERAAEELKKSPDPEAQKEAKKAEQKVRCERPGS
>2DS25_DHR82C 86.4%
((SEQ ID NO: 110)
DEEEIQKAIEELLEKGVSEEEAAIIIVQR
FNVAVVVVVQDEREGKEISEEIRKAIPDA
DVILFANLVVIKVETHELSTVVWLIAQGLM
LLLAIKQNPDNDRAVEEAVRVARKLKKLAEELQEKAKKTGDAKLLTLALT
LLLFAVKLVELAIKSNPDNDEAVETAVRLARELKKVAEELQERAKKTGDA
ELLKLALEALEVAVRAVELAIKSNPDNEEAVETAKRLAEELRKVAELLEE
RAKETGDPELQELAKRAKEVADRARELAKCSNPNN

In some embodiments, a given amino acid can be replaced by a residue having similar physiochemical characteristics. e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are known. Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G). Ser (S), Thr (T), Cys (C), Tyr (Y). Asn (N), Gln (Q); (3) acidic: Asp (D), Glu (E): (4) basic: Lys (K), Arg (R), His (H). Alternatively, naturally occurring residues can be divided into groups based on common side-chain properties: (1) hydrophobic: Norleucine, Met. Ala, Val, Leu, lie: (2) neutral hydrophilic: Cys, Ser, Thr, Asn, Gln: (3) acidic: Asp, Glu: (4) basic: His, Lys. Arg: (5) residues that influence chain orientation: Gly. Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into H is; Asp into Glu; Cys into Ser: Gln into Asn; Glu into Asp; Gly into Ala or into Pro: His into Asn or into Gin: Ile into Leu or into Val; Leu into Ile or into Val; Lys into Arg, into Gln or into Glu: Met into Leu, into Tyr or into lie: Phe into Met, into Leu or into Tyr; Ser into Thr; Thr into Ser; Trp into Tyr; Tyr into Trp; and/or Phe into Val, into Ile or into Leu.

In all of these embodiments, the percent identity requirement does not include any additional functional domain that may be incorporated in the polypeptide.

In another embodiment, the transferrin receptor binding polypeptides of the disclosure may bind to the transferrin receptor with a binding affinity of at least 3 μm, 1 μm, 500 nm, 250 nm, 100 nm, or 50 nm.

In another aspect, the disclosure provides recombinant nucleic acid encoding the polypeptide of any embodiment or combination of embodiments disclosed herein the can be generically encoded. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded polypeptide, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the polypeptides of the disclosure.

In another aspect, the disclosure provides expression vectors comprising the recombinant nucleic acid of the disclosure operatively linked to a promoter. “Expression vector” includes vectors that operatively link a nucleic acid coding region or gene to any control sequences capable of effecting expression of the gene product. “Control sequences” operatively linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In one aspect, the disclosure provides recombinant host cell comprising the polypeptide, nucleic acid, and/or the expression vector (episomal or chromosomally integrated) of any embodiment disclosed herein. The host cells can be either prokaryotic or eukaryotic.

In another aspect, the disclosure provides pharmaceutical compositions, comprising the polypeptide, the recombinant nucleic acid, the expression vector, or the recombinant host cell of any of any embodiment or combination of embodiments, and a pharmaceutically acceptable carrier. The pharmaceutical compositions of the disclosure can be used, for example, in the methods of the disclosure described herein. The pharmaceutical composition may further comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The pharmaceutical composition may also include a lyoprotectant, e.g. sucrose, sorbitol or trehalose. In certain embodiments, the pharmaceutical composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the pharmaceutical composition includes a bulking agent, like glycine. In yet other embodiments, the pharmaceutical composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The pharmaceutical composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the pharmaceutical composition additionally includes a stabilizer, e.g., a molecule which, when combined with a protein of interest substantially prevents or reduces chemical and/or physical instability of the protein of interest in lyophilized or liquid form. Exemplary stabilizers include sucrose, sorbitol, glycine, inositol, sodium chloride, methionine, arginine, and arginine hydrochloride.

The polypeptide, nucleic acid, expression vector, or cell of any embodiment or combination of embodiments herein may be the sole active agent in the pharmaceutical composition, or the composition may further comprise one or more other active agents suitable for an intended use.

In a further aspect, the disclosure provides methods for using, or a use of the polypeptide, the recombinant nucleic acid, the expression vector, the recombinant host cell, and/or the pharmaceutical composition of any embodiment or combination of embodiments of the disclosure, for any suitable purpose including but not limited to those disclosed herein.

In various embodiments, the purpose includes, but is not limited to, treating or limiting arenavirus infection; delivery of therapeutics for treating tumors; and fusion to therapeutics such as biologicals (including but not limited to protein, nucleic acid, and antibody therapeutics) to increase serum half-life of the therapeutic.

The TfR apical domain (where the polypeptides of the disclosure bind, as discussed in the examples that follow) also serves as the site for the entry of new world arenaviruses into cells (Abraham et al. 2010, Nat. Struct. Mol. Biol. 17, 438-444 (2010); Clark et al. 2018; Nat. Commun. 9, 1884 (2018).). A number of these viruses such as Machupo, Junin, Guanarito and Sabiá viruses cause hemorrhagic fevers with high fatality rates. Hence, the polypeptides of the disclosure may block viral entry the same way antibodies that bind to the apical domain can block viral entry.

TfR is overexpressed in a number of tumors (Daniels-Wells, T. R. and Penichet, M. L. Transferrin receptor 1: a target for antibody-mediated cancer therapy. Immunotherapy 8, 991-994 (2016)) raising the possibility of targeted therapy using the disclosed polypeptides as targeting module. Similarly since, TfR is expressed throughout the body, the disclosed polypeptides may be exploited as a general delivery platform.

Finally TfR binding proteins have been suggested to be useful as recycling factors to increase the lifetime of biologics in serum. Like the Fc receptor, TfR continuously cycling between the cell surface and endocytotic vesicles as part of its natural function to deliver serum Tf into cells. Fusion of biologics to Tf has increased their serum lifetime. Fusions of biologics to the disclosed polypeptides could likewise lead to increased lifetime of the biologic.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

### Examples

The de novo design of polar protein-protein interactions is challenging because of the thermodynamic cost of stripping water away from the polar groups. Here we describe a general approach for designing proteins which complement exposed polar backbone groups at the edge of beta sheets with geometrically matched beta strands, forming a beta sheet extension. We applied our protocol to the computationally design small proteins which bind to an exposed beta sheet on the human Transferrin Receptor (hTfR) which shuttles interacting proteins across the Blood-Brain-Barrier (BBB), opening up new avenues for drug delivery into the brain. Our designed BBB shuttle protein binds hTfR with nanomolar affinity, is hyperstable and crosses the BBB in an in vitro microfluidic organ-on-a-chip model of the human BBB.

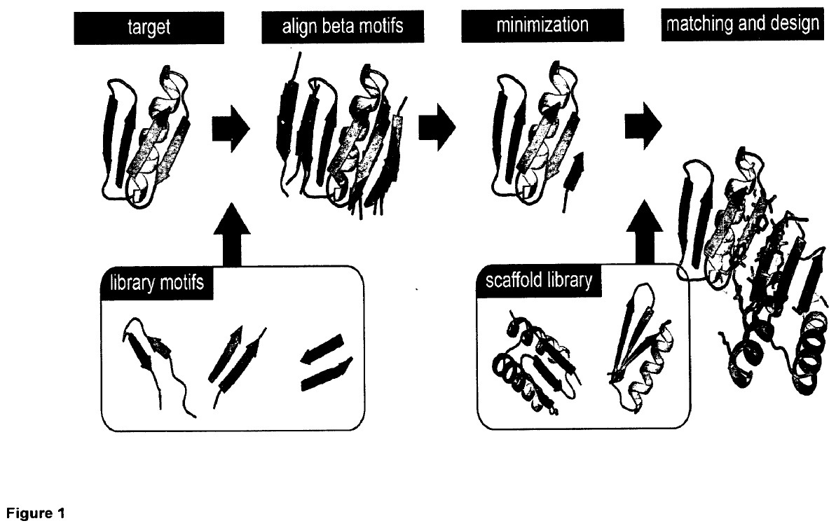

While most protein-protein interfaces are composed primarily of sidechain-sidechain interactions, backbone hydrogen bonding can also play a role. We developed a computational design approach for designing binding proteins with beta sheets geometrically poised to pair with exposed beta strands in target proteins of interest. We first align short 2-stranded beta sheets or beta hairpins to the target protein edge strands and then use gradient based minimization of the backbone coordinates to optimize the hydrogen bonding interactions across the interface with the target (FIG. 1a). These optimized beta strands are then grafted onto de novo designed small protein scaffolds with geometrically matching beta sheets, yielding a docked protein-protein complex. After filtering docks based on hydrogen bond geometry and buried surface area across the interface, Rosetta™ flexible backbone combinatorial sequence optimization is used to maximize the sidechain-sidechain interaction energy and the stability of the designed scaffold.

### Design of a Human Transferrin Receptor Binding Protein

We sought to use our protocol to design a human Transferrin Receptor (hTfR) binding protein, hTfR transports transferrin (the major carrier of iron in the body) across the BBB via receptor mediated transcytosis, and this process has been exploited to deliver therapeutic payloads into the brain parenchyma that would otherwise be blocked by the BBB. For example, antibodies and nanoparticles linked to larger complicated molecules such as Transferrin or anti-TfR antibodies have been shown to cross the BBB into the brain parenchyma in a hTfR dependent manner. Thus, hTfR is an attractive target candidate for the development of BBB traversing vehicles.

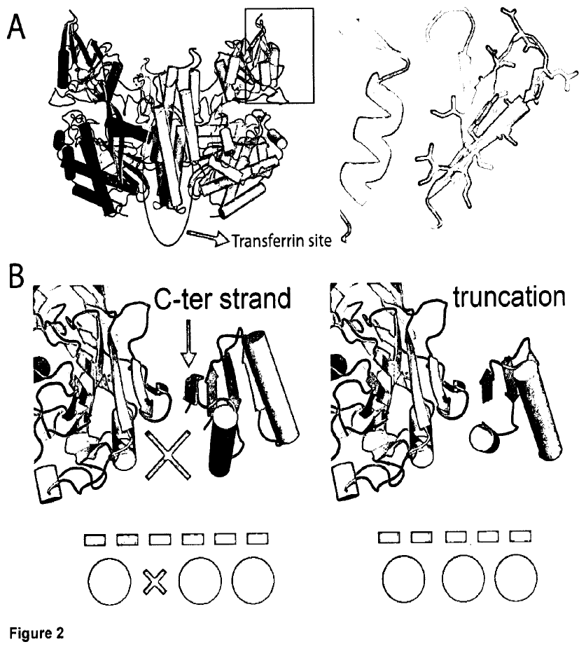

We aimed to design binders to the hTfR outside of the transferrin binding site to avoid competition with transferrin. The apical domain of the TfR contains an exposed edge strand suitable for beta sheet extension⁸, and we applied our design protocol to this region (FIG. 2a).

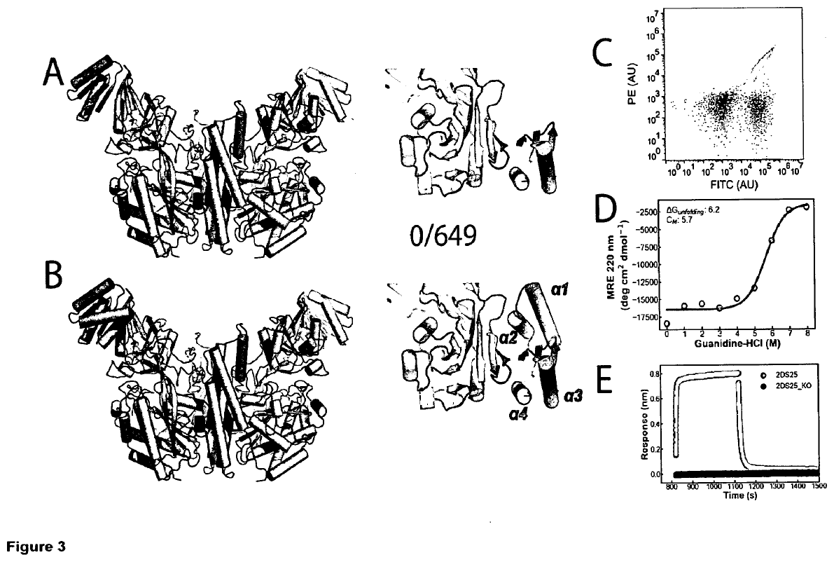

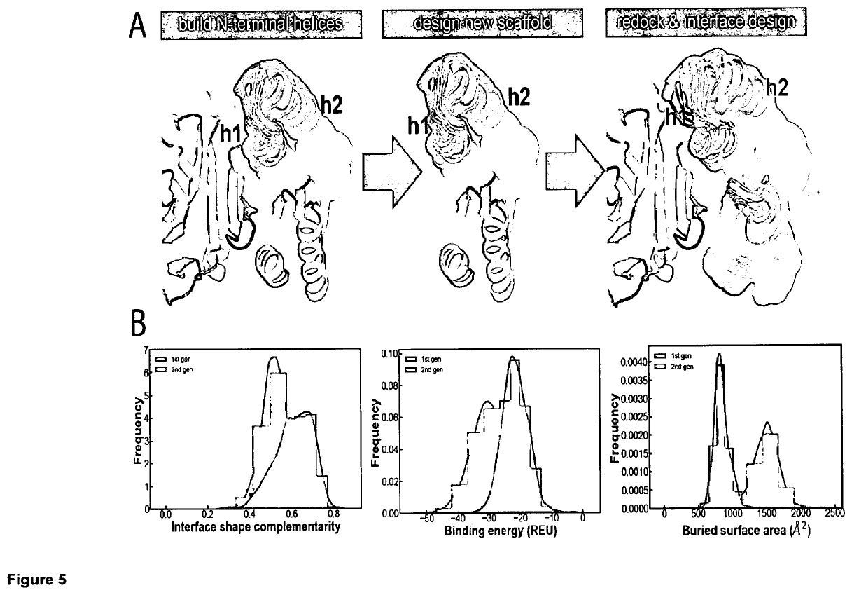

In the strand matching step, we found that a C-terminally truncated version of a de novo designed ferredoxin scaffold could bury substantial surface area and make excellent beta sheet hydrogen bond interactions across the interface (FIG. 2b). The sequences of the docked scaffolds were optimized for high affinity interactions, and a library of 649 selected designs were ordered on an oligoarray and tested for binding using yeast surface display. However, none of these designs bound hTfR despite having high in silico folding propensity and high interface shape complementarity (FIG. 3a and FIG. 5b).

We hypothesized that the flaw in these first round designs was the low interface buried surface area, which ranged from 144 Å² to 1395 Å², but averaged only 842 Å². To test this hypothesis, we used RosettaRemodel™ to expand the starting scaffold by adding a new poly valine helix at the N-terminus to form a second interface with the target. Thousands of new backbones were generated, in some of which the secondary interface helix was stabilized with another buttressing helix (FIG. 5a). After generating the backbones and using Rosetta™ combinatorial design calculations to optimize the sequences, the lowest energy scaffolds were redocked to hTfR, and the interface residues again optimized for tight binding (FIG. 3b). These second round designs had greater buried surface area with the target while retaining good shape complementarity (FIG. 5b).

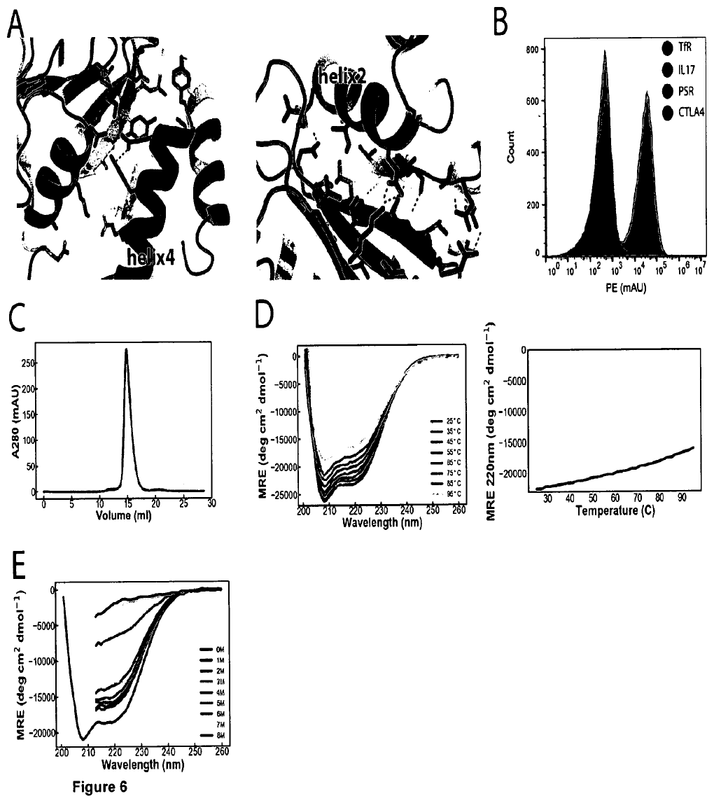

Synthetic genes encoding 50 designs were obtained and hTfR binding was tested using yeast surface display. Of the 50, one (designated 2DS25) clearly bound fluorescently labeled hTfR (FIG. 3c). In the design model, two helices on either side of the central beta sheet extension make contacts with TfR across the interface (FIG. 6a). Binding was specific as 2DS25 did not bind to the edge strand containing proteins CTLA4 and IL17 nor to polyspecificity reagents developed previously for the identification of nonspecific antibodies (FIG. 6b).

We next expressed and purified 2DS25 from E. coli using immobilized metal affinity chromatography. The protein eluted as a monodisperse peak from size exclusion chromatography at an elution volume that corresponds to a monomer (FIG. 6c). Circular dichroism spectroscopy showed that 2DS25 is highly stable: the melting temperature is above 95 degrees, and the Guanidine-HCl concentration required for 50% denaturation was 5.7M (FIG. 3d and FIG. 6d-e). Purified 2DS25 bound the hTfR ectodomain in biolayer interferometry experiments (FIG. 3e). Mutation of key residues in the designed binding site abolished binding, suggesting that complex formation is through the designed interface (FIG. 3e).

To probe the sequence determinants of folding and binding, and to facilitate determination of the structure of the 2DS25-hTfR complex, we created a site saturation mutagenesis library (SSM) in which each position on 2DS25 was substituted with all other twenty amino acids one at a time, and screened for hTfR binding using FACS. Deep sequencing revealed that the designed core residues of 2DS25 were conserved suggesting 2DS25 folds as designed. The key interface residues were also conserved while affinity increasing substitutions were identified around the interface. Combination of these enriched substitutions yielded higher affinity variants (see methods).

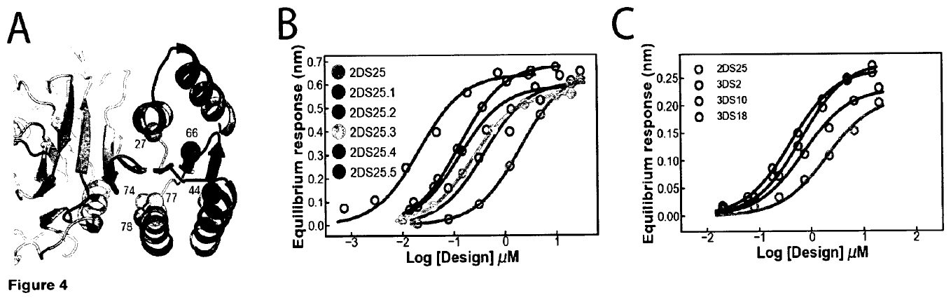

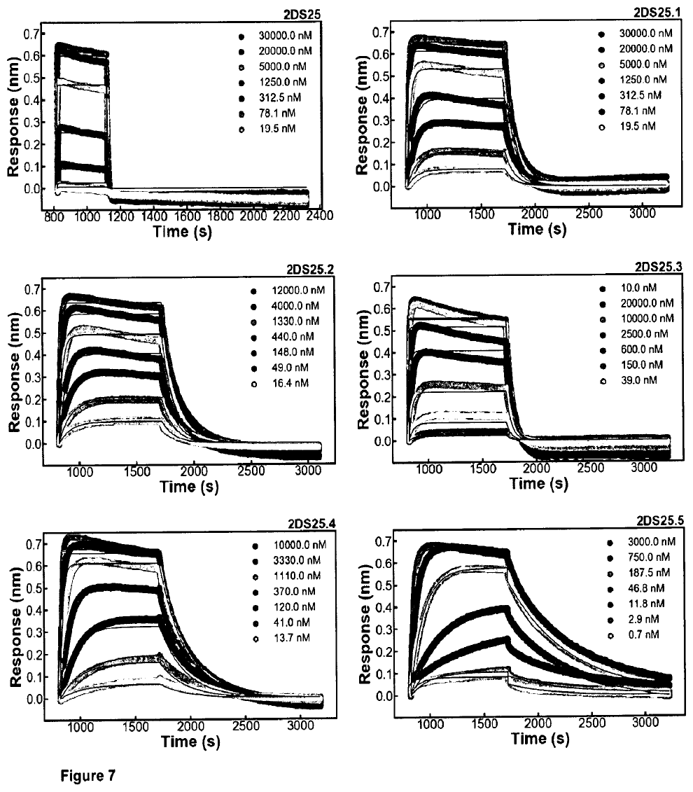

Binding affinity is a key factor determining transcytosis efficiency of compounds targeting hTfR. We took advantage of the SSM data to create a range of designs with different K_{D}'s to test for BBB traversal. The majority of the mutants that improved binding map to the interface between hTfR and 2DS25 and likely optimize packing interactions and electrostatic contacts (FIG. 4a,). Two mutants (A44G and 166L) that improved binding occurred in the core of 2DS25 distal to the interface; these may produce subtle conformational alterations that stabilize the interface. Biolaver interferometrv of 5 variants revealed Kds ranging from 20 nM to 400 nM (FIG. 4b and FIG. 7).

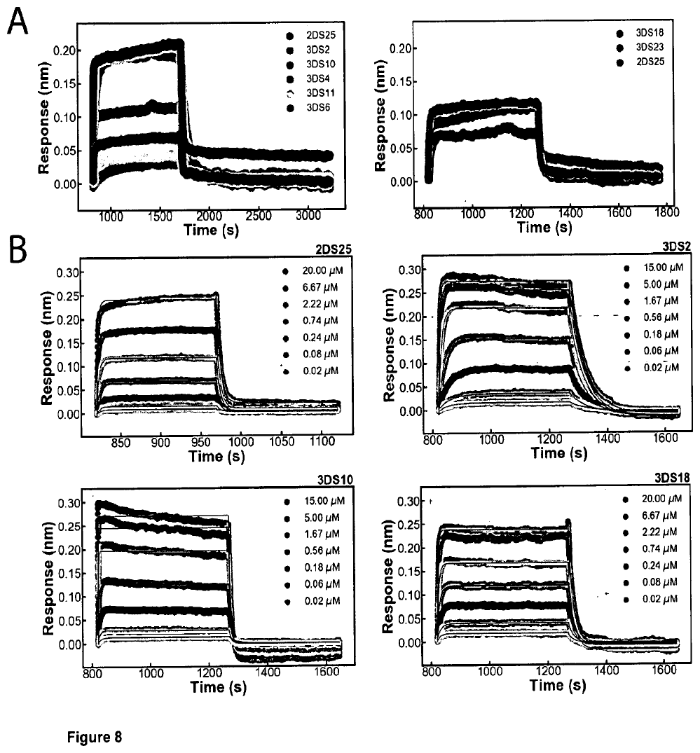

Based on the above results and structural analyses, we performed another round of design. We selected 48 designs and expressed them in E. coli. Of the 48 designs ordered 24 were soluble after SEC and 7 designs showed binding signal in biolayer interferometry (FIG. 8a), a more than 7-fold improvement in success rate compared to the previous design round. We proceeded with 3 designs for further biophysical characterization and found that they bound with affinities ranging from 400-700 nM (FIG. 4c and FIG. 8b).

### Discussion

Our method for computationally designing small proteins that bind to exposed beta strands and neighboring regions on protein targets considerably expands the possibilities for protein inhibitor design. “One sided” interface design in which a protein is de novo designed to bind to a fixed target protein with high specificity and affinity has been largely limited until now to targets with surface hydrophobic patches which can be complemented by appropriately shaped hydrophobic clusters on the designed protein. Our method now makes available the much more polar and less concave regions surrounding edge beta strands, and hence increases the number of proteins of interest which can be targeted. The advantage of computational design over antibody and other selection methods in being able to choose the region of the target being bound is clear in the hTfR case; we selected a site far away from the transferrin binding site to avoid competition.

Our small stable designed hTfR binder, and similar designs against other targets at the BBB, provide exciting new possibilities for transporting therapeutics and other molecular cargo into the brain. The small size (10 kDa) offers improved access to the brain via receptor mediated transcytosis compared to antibodies and the cognate ligand Transferrin (which is 76 10 kDa). Given the high stability and modularity, and hence robustness to genetic fusion and chemical coupling, our designs have a distinct advantage over larger more complicated molecules for fusion/coupling to therapeutic cargoes.

### Materials & Methods

### Protein Design

Identification of Target Edge Strands The Transferrin receptor target protein (pdb 3kas) was relaxed into the Rosetta™ energy function using coordinate constraints after removing HETATM records. All target protein edge strands were identified visually by inspection in a molecular graphics viewer, or programmatically by calculating the atomic solvent accessible surface area (aSASA) of all backbone H and O atoms present in residues that were in beta conformation. Strands with a length of at least 3 residues and an average aSASA value above 2 were considered solvent exposed, and hence, edge strands suitable for strand docking.

### Geometric Matching Beta Motifs to Edge Strands

The C-alpha atoms of computationally generated beta hairpin motifs, and short parallel and antiparallel 2 stranded beta sheets derived from the PDB were aligned onto the target edge strand. The aligned segment of the motifs were next deleted. The docked strands were then either trimmed down further or extended at either the N or C terminus, creating a range of strands with different lengths. These docks were relaxed using gradient-descent-based minimization in presence of the target using Rosetta™ FastRelax™ to optimize backbone hydrogen bond interactions with the target edge strand. Docks failing a specified threshold value (typically −4) for the backbone hydrogen bond scoreterm in Rosetta™ (hbond_Ir_bb) were discarded.

Matching Docked and Minimized Strands into Scaffolds

Strands were geometrically matched with our scaffold library using the MotifGraftMover™ in Rosetta™. Following matching the resulting protein-protein complexes were repacked at the interface using the PackRotamersMover™ followed by cartesian and kinematic (FastRelax) minimization to regularize the potentially broken bonds at the junctions of the docked strand and the scaffold. For the heterodimers, only docks that buried an interface of at least 1100 Å² were selected for downstream design rounds.

### Interface Design and Filtering

The interface side chains of the complexes were designed using Rosetta™ combinatorial sequence optimization with as score function “ref2015” or “beta_nov16” or “beta_genpot” to maximize the sidechain-sidechain interaction energy and the stability of the designed scaffolds. During sequence optimization, the backbones of the designed scaffolds were allowed to move enabling finer sampling of the possible side chains. In addition, rigid body minimization was allowed during the design protocol. The amino acid identities of the explicit hydrogen bond networks present in heterodimers were fixed and constrained to their original atomic positions during sequence optimization, and only allowed to move during a final minimization step.

In general, the best designs in terms of interface energy per buried surface area (<=-25 Rosetta Energy Units (REU)), interface shape complementarity (>=0.6), interface buried surface area (>=1200 Å²), average per residue energy (<=−2 REU) and number of buried unsatisfied polar in atoms in the interface (<=3) were inspected visually before selecting designs for ordering as synthetic genes. For the hTfR binders as an additional filtering step, multiple independent Rosetta™ folding simulations were performed to assess whether our designed sequences would fold into the lowest energy structures without off-target minima.

### Backbone Generation and Scaffold Design

De novo designed ferredoxin-like scaffolds that served as the basis for the first hTfR binders were modified and expanded using blueprint based backbone generation. Backbone generation was biased to only include idealized canonical loops to connect secondary structure elements. Rosetta™ combinatorial sequence optimization was used to design the sequence of the new backbones. Low energy designs that folded into the designed structure in Rosetta folding simulations were selected and used as scaffolds for hTfR binders.

### Protein Purification and Expression

Synthetic genes encoding designed proteins and their variants were purchased from IDT DNA technologies or Genscript. Sequences included N-terminal histidine tags followed by a TEV cleavage site. All genes were expressed by autoinduction in TBII media (Mpbio) supplemented with 50×5052, 20 mM MgSO4 and trace metal mix. Expression was allowed under antibiotics selection at 37 degrees overnight or at 18-25 degrees overnight after initial growth for 6-8 h at 37 degrees.

Next, cells were harvested by centrifugation and lysed by sonication after resuspension of the cells in lysis buffer (100 mM Tris pH 8.0, 200 mM NaCl, 50 mM Imidazole pH 8.0) containing protease inhibitors (Thermo Scientific) and Bovine pancreas DNaseI (Sigma-Aldrich). Proteins were subsequently purified by Immobilized Metal Affinity Chromatography. Cleared lysates were applied to 2-4 ml nickel NTA beads (Qiagen) and incubated in batch for 20 minutes before washing beads with 10-20 column volumes of lysis buffer. Designs were eluted in elution buffer (20 mM Tris pH 8.0, 100 mM NaCl, 500 mM Imidazole pH 8.0) after which the histidine tags were cleaved using histidine tagged TEV protease while dialyzing against dialysis buffer overnight (20 mM Tris pH8.0, 100 mM NaCl). A second IMAC purification was performed the next day for TEV cleaved samples to capture uncleaved protein and TEV protease. Designs were finally polished using size exclusion chromatography (SEC) on either Superdex™ 200 Increase 10/300GL or Superdex™75 Increase 10/300GL columns (GE Healthcare) using SEC buffer (10 mM HEPES pH 7.5, 100 mM NaCl). Peak fractions were verified by SDS-PAGE and LC/MS and stored at concentrations between 1-10 mg/ml at 4 degrees or flash frozen in liquid nitrogen for storage at −80.

The human transferrin receptor 1 ectodomain (uniprot P02786-I) was expressed as a fusion protein (IgK-sFLAG-His-Sen-TEV-TfR 1-his-Avin) using the Daedalus expression system²⁰. After cleaving the N-terminal expression tag with TEV, the protein was further purified by SEC. Peak fractions were biotinylated using an in vitro biotinylation kit (Avidity). Biotinylated TfR was further purified by Superdex™200 Increase 10/300GL in SEC buffer. Peak fractions were concentrated to ˜1.5 mg/ml, flash-frozen and stored at −80 degrees.

### Circular Dichroism

CD spectra were recorded on a J-1500 instrument (Jasco, Easton, Md.) in a 1 mm path length cuvette at a protein concentration of 0.32 mg/ml (chemical melts) or 0.4 mg/ml (temperature melts). For temperature melts, data was recorded at 220 nm between 25 and 95° C. every 2 degrees, and wavelength scans (190-260 nm) were recorded every 10° C. in DPBS buffer (Gibco). Chemical denaturation wavelength scans were recorded between 190-260 nm in the presence of Guanidine-HCl buffer at 25° C. Data recorded at 220 nm during the chemical denaturation melts were fitted to the following model²¹ using custom python scripts to obtain the m-value, ΔG_{O}, S_{N}, S_{D} and midpoint of denaturation value (C_{M}).

$$fD = 1 / 1 + {e}^{\frac{m [ denaturant ] - Δ {G}_{0}}{RT}}$$

$$S = {S}_{N} + ( {S}_{D} - {S}_{N} ) fD$$

where S in the observed signal, S_{N} the signal of the folded baseline, and S_{D} the signal of the denatured baseline. C_{M} was obtained by

$${C}_{M} = \frac{Δ {G}_{0}}{m}$$

### Library Generation

The gene library for the first generation hTfR binders was ordered from Agilent Technologies with flanking adaptor sequences to allow amplification of the genes, qPCR using Kapa HiFi Hotstart™ Ready Mix (Kapa Biosystems) was performed to amplify the library in order to prevent overamplification that would reduce transformation efficiency. After amplification and DNA gel electrophoresis, DNA was purified using a gel extraction kit (Qiagen) and subjected to a second qPCR amplification round to add pETCON™ adaptors to both DNA ends to facilitate cloning into the yeast surface display vector pETCON™. This gene pool was again purified by gel extraction.

The 2DS25 Site Saturation Mutagenesis library was generated by overlap extension PCR at each codon of the 2DS25 gene. Randomized primers were purchased from Integrated DNA Technologies. After verification of desired inserted size by DNA gel electrophoresis, a 2nd PCR was performed to add pETCON™ adaptors to both DNA ends to facilitate cloning. For both libraries EBY100 electrocompetent yeast cells were transformed by electroporation with the linear library DNA together with the linearized (NdeI/XhoI) pETCON™ yeast surface display vector as described earlier²².

### Yeast Surface Display and Deep Sequencing

Myc tagged designs were displayed on the yeast surface as Aga2p fusion proteins. The diversity of the libraries was below 10⁶ in all cases. Yeast cells were grown at 30° C. in C-trp-ura+2% glucose media for 16-24 h before expression was induced by transferring cells to SGCAA media for 16-24 h at 30° C. Cells were harvested by centrifugation and washed twice with PBSF (PBS supplemented with 1% bovine serum albumin). Cells were subsequently incubated with biotinylated target for 0.5-2 h at room temperature before being washed twice with PBSF. These cells were next labeled with streptavidin-phycoerythrin and a FITC conjugated anti-Myc antibody (ICL Lab) for 20 minutes before being washed again. For initial screening for binding signals, biotinylated target was pre-incubated with streptavidin-phycoerythrin (Invitrogen) for 10 minutes before the complex was added to cells enabling the identification of weak binders by using avid binding conditions. Samples were sorted or measured in a Sony SH800 cell sorter or Accuri™ flow cytometer (BD biosciences) using the FITC and phycoerythrin (PE) signals. Sorted cells were collected and grown in C-trp-ura+2% glucose media for 24-48 h before being frozen at −80° C. for later analyses. SSM libraries were selected against 100 nM, 20 nM and 7 nM of hTfR whereas the combination libraries were selected against 250 nM, 10 nM, 1 nM, 0.5 nM, 0.250 nM and 0.125 nM hTfR.

DNA preparation for deep sequencing was performed as described before²³. DNA was sequenced using MiSeq™ sequencer with a 600-cycle reagent kit (Illumina). Reads were aligned with PEART software²⁴. Sequences were finally analyzed using custom scripts based on the Enrich™ software²⁵.

### Combination Variants Generation

After deep sequencing analyses of the site saturation mutagenesis library we identified 13 positions where individual mutations improved binding. Two approaches were followed to further optimize the binding affinity. First a subset of selected mutants were manually combined and ordered as synthetic genes for testing in binding assays. This approach yielded 2DS25.3.

In the second approach we generated a combination library. We ordered two overlapping Ultramer™ oligonucleotides (Integrated DNA Technologies), containing degenerate codons for the 13 specified positions. Ultramer™ fragments were assembled and PCR amplified before being electroporated as described above. After selecting the best binders in yeast surface display by Sanger sequencing, designs were ordered as synthetic genes and purified for testing in biolayer interferometry binding assays.

Surprisingly, high affinity variants on the yeast surface only bound with moderate affinity in the biolayer interferometry assays. Even though the off-rate decreased in these variants, this decrease was generally accompanied by a compensatory decrease in on-rate. In order to create high affinity variants with fast on-rates and slower off-rates we manually combined positions of the SSM, 2DS25.3 and combination library mutants yielding 2DS25.5.

### Biolayer Interferometry

Binding assays were performed on an OctetRED96™ BLI system (ForteBio, Menlo Park, Calif.) using streptavidin-coated biosensors. Biosensors were equilibrated for at least 10 minutes in Octet™ buffer (10 mM HEPES pH 7.4, 150 mM NaCl, 3 mM EDTA, 0.05% Surfactant P20) supplemented with 1 mg/mI Bovine Serum Albumin (SigmaAldrich). For each experiment the biotinylated hTfR ectodomain was immobilized onto the biosensors by dipping the biosensors into a solution with 10-50 nM hTfR for 200-500s. Followed by dipping in fresh octet buffer to establish a baseline for 200 s in buffer. Titrations were executed at 25° C. while rotating at 1,000 r.p.m. Association of designs to TfR on the biosensor was allowed by dipping biosensors in solutions containing designed protein diluted in octet buffer for 900 s. After reaching equilibrium, the biosensors were dipped into fresh buffer solution in order to monitor the dissociation kinetics for 900-1500 s. In single concentration assays, 1 μM of design was used diluted in Octet buffer. For equilibrium binding titrations, kinetic data were collected and processed using a 1:1 binding model to obtain the equilibrium binding response Req using the data analysis software 9.1 of the manufacturer. Multiple binding experiments with different protein preparations under different hTfR immobilization densities to ensure reproducibility. Representative binding curves are presented in the main text. For each design seven Req values were fitted with a custom python script to a saturation binding curve to obtain Bm. and the equilibrium dissociation constant K_{D}.

$$Y = \frac{{B}_{max} \cdot X}{{K}_{D} + X}$$

### REFERENCES

1. Remaut, H. & Waksman, G. Protein-protein interaction through beta-strand addition. Trends Biochem. S. 31, 436-444 (2006).
2. Watkins, A. M. & Arora, P. S. Anatomy of r-strands at protein-protein interfaces. ACS Chem. Biol. 9, 1747-1754 (2014).
3. Chevalier, A. et al. Massively parallel de novo protein design for targeted therapeutics. Nature 550, 74-79 (2017).
4. Lajoie, J. M. & Shusta, E. V. Targeting receptor-mediated transport for delivery of biologics across the blood-brain barrier. Annu. Rev. Pharmacol. Toxicol. 55, 613-631 (2015).
5. Yu, Y. J. et al. Therapeutic bispecific antibodies cross the blood-brain barrier in nonhuman primates. Sci. Transl. Med. 6, 261ra154 (2014).
6. Yu, Y. J. et. al. Boosting brain uptake of a therapeutic antibody by reducing its affinity for a transcytosis target. Sci. Transl. Med. 3, 84ra44 (2011).
7. Clark, A. J. & Davis, M. E. Increased brain uptake of targeted nanoparticles by adding an acid-cleavable linkage between transferrin and the nanoparticle core. Proc. Natl. Acad. Sci. U.S.A 112, 12486-12491 (2015).
8. Abraham, J., Corbett, K. D., Farzan, M., Choe, H. & Harrison, S. C. Structural basis for receptor recognition by New World hemorrhagic fever arenaviruses. Nat. Struct. Mot. Biol. 17, 438-444 (2010).
9. Chen, J., Sawyer, N. & Regan, L. Protein-protein interactions: general trends in the relationship between binding affinity and interfacial buried surface area. Protein Sci. 22, 510-515 (2013).
10. Huang, P.-S. et (a. RosettaRemodel: a generalized framework for flexible backbone protein design. PLoS One 6, e24109 (2011).
11. Fleishman, S. J. et al. RosettaScripts: a scripting language interface to the Rosetta macromolecular modeling suite. PLoS One 6, e20161 (2011).
12. Xu, Y. et al. Addressing polyspecificity of antibodies selected from an in vitro yeast presentation system: a FACS-based, high-throughput selection and analytical tool. Protein Eng. Des. Sel. 26, 663-670 (2013).
13. Niewoehner, J. et al. Increased brain penetration and potency of a therapeutic antibody using a monovalent molecular shuttle. Neuron 81, 49-60 (2014).
14. Lin, Y.-R. et al. Control over overall shape and size in de novo designed proteins. Proc. Natl. Acad. Sci. U.S.A 112, E5478-85 (2015).
15. Hosseinzadeh, P. et al. Comprehensive computational design of ordered peptide macrocycles. Science 358, 1461-1466 (2017).
16. Bhardwaj, G. et al Accurate de novo design of hyperstable constrained peptides. Nature 538, 329-335 (2016).
17. Rocklin. G. J. et al. Global analysis of protein folding using massively parallel design, synthesis, and testing. Science 357, 168-175 (2017).
18. Dang, B. et al. De novo design of covalently constrained mesosize protein scaffolds with unique tertiary structures. Proc. Natl. Acad. Sci. U.S.A 114, 10852-10857 (2017).
19. Khatib. F. et al. Algorithm discovery by protein folding game players. Proc. Natl. Acad. Sci. USA. 108, 18949-18953 (2011).
20. Bandaranayake, A. D. et al Daedalus: a robust, turnkey platform for rapid production of decigram quantities of active recombinant proteins in human cell lines using novel lentiviral vectors. Nucleic Acids Res. 39, e143 (2011).
21. Myers, J. K., Pace, C. N. & Scholtz, J. M. Denaturant m values and heat capacity changes: relation to changes in accessible surface areas of protein unfolding. Protein Sci. 4, 2138-2148 (1995).
22. Procko, E. et al. Computational design of a protein-based enzyme inhibitor. J Mol. Biol. 425, 3563-3575 (2013).
23. Berger, S. et al. Computationally designed high specificity inhibitors delineate the roles of BCL2 family proteins in cancer. Elife 5, (2016).
24. Zhang, J., Kobert, K., Flouri, T. & Stamatakis, A. PEA a fast and accurate Illumina Paired-End reAd mergeR. Bioinformatics 30, 614-620 (2014).
25. Fowler, D. M., Araya, C. L., Gerard. W. & Fields. S. Enrich: software: for analysis of protein function by enrichment and depletion of variants. Bioinformatics 27, 3430-3431 (2011).

### Details of the Tested Transferrin Receptor Binding Proteins

2DS25 type (Sequences Described Above)

2DS25 variants are single point mutants based off 2DS25. The point mutants improve TfR binding. All 2DS25 type design have the same topology, length and structure. Positions are equivalent i.e. position 27 in 2DS25 has the same location in Cartesian space as 2DS25.6 but the amino acid identity at the position may differ between variants

### Third Generation 3DS Type Binders (Sequences Described Above)

These designs are based off the 2DS25 type designs and hence have the same secondary structure organization and binding mode as the 2DS25 type designs. Elements and residues directly contacting TfR are in H2, E1 and H4.

1. A transferrin receptor binding polypeptide comprising the general formula H1-H2-E1-H3-E2-E3-H4, wherein
H1, H2, H3, and H4 each independently comprise an alpha helical domain of between 11-20 amino acids in length;
E1, E2, and E3 each independently comprise a beta sheet of 5 amino acids in length; and
optional amino acid linkers between one or more domains;
wherein the polypeptide binds to the transferrin receptor.

2. (canceled)

3. The transferrin receptor binding polypeptide of claim 1, wherein H1 comprises an amino acid sequence at least 60% identical to the amino acid sequence selected from the group consisting of SEQ ID NO: 1-8 and 86.

4. The transferrin receptor binding polypeptide of claim 1, wherein at least 40%, 50%, or 60% of residues in H2 are hydrophobic.

5. (canceled)

6. The transferrin receptor binding polypeptide of claim 1, wherein H2 comprises an amino acid sequence at least 60% identical to the amino acid sequence selected from the group consisting of SEQ ID NO: 9-18 and 87.

7-8. (canceled)

9. The transferrin receptor binding polypeptide of claim 1, wherein H3 comprises an amino acid sequence at least 60% identical to the amino acid sequence selected from the group consisting of SEQ ID NO: 19-27 and 88-92.

10. (canceled)

11. The transferrin receptor binding polypeptide of claim 1, wherein H4 comprises an amino acid sequence at least 60% identical to the amino acid sequence selected from the group consisting of SEQ ID NO: 28-39 and 93-97.

12. (canceled)

13. The transferrin receptor binding polypeptide of claim 1, wherein the polypeptide comprises an amino acid sequence at least 60% identical to the amino acid sequence of H1, H2, H3, and H4 domains from a single row selected from rows (a)-(t) of Table 1.

14. (canceled)

15. The transferrin receptor binding polypeptide of claim 1, wherein at least 3, 4, or all 5 of the amino acids in each of E1, E2, and E3 are hydrophobic.

16. The transferrin receptor binding polypeptide of claim 1, wherein
(a) E1 comprises the amino acid sequence of SEQ ID NO:63;
(b) E1 comprises the amino acid sequence selected from the group consisting of SEQ ID NO. 40-45;
(c) E2 comprises the amino acid sequence of SEQ ID NO:64;
(d) E2 comprises the amino acid sequence selected from the group consisting of SEQ ID NO: 46-53 and 98;
(e) wherein E3 comprises the amino acid sequence of SEQ ID NO:65; and/or
(f) E3 comprises the amino acid sequence selected from the group consisting of SEQ ID NO. 54-62.

17-21. (canceled)

22. The transferrin receptor binding polypeptide of claim 1, wherein the E1, E2, and E3 domains comprise an amino acid sequence at least 60% identical to the amino acid sequence of E1, E2, and E3 domains from a single row of selected from rows (a)-(o) of Table 2, wherein amino acid substitutions relative to the reference domain are conservative amino acid substitutions.

23. The transferrin receptor binding polypeptide of claim 1, comprising amino acid linkers between one or more adjacent domains.

24. (canceled)

25. The transferrin receptor binding polypeptide of claim 1, wherein the polypeptide comprises an amino acid sequence at least 50% to the amino acid sequence selected from the group consisting of SEQ ID NO: 66-85.

26. The transferrin receptor binding polypeptide of claim 1, comprising one or more additional functional domains.

27-31. (canceled)

32. The transferrin receptor binding polypeptide of claim 26, comprising an amino acid sequence at least 50% identical to the amino acid sequence selected from the group consisting of SEQ ID NO: 105-110.

33. The transferrin receptor binding polypeptide of claim 1, wherein the polypeptides bind to the transferrin receptor with a binding affinity of at least 3 μm.

34. A recombinant nucleic acid encoding the polypeptide of claim 1.

35. An expression vector comprising the recombinant nucleic acid of claim 34 operatively linked to a promoter.

36. A recombinant host cell comprising the expression vector of claim 35.

37. A pharmaceutical composition, comprising the polypeptide of claim 1, and a pharmaceutically acceptable carrier.

38. A method for using, the polypeptide of claim 1 for any suitable purpose including but not limited to those disclosed herein.

39. (canceled)
